(19) United States
(12) Patent Application Publication
(10) Pub. No.: US 2018/0296588 A1
(43) Pub. Date: Oct. 18, 2018

(54) COMBINATION THERAPY FOR TREATING VIRAL INFECTIONS

(71) Applicant: ContraVir Pharmaceuticals, Inc., Edison, NJ (US)

(72) Inventors: Robert Thomas FOSTER, Edmonton (CA); Daniel Joseph TREPANIER, Edmonton (CA); Daren Raymond URE, Edmonton (CA)

(21) Appl. No.: 15/952,271

(22) Filed: Apr. 13, 2018

Related U.S. Application Data
(60) Provisional application No. 62/485,510, filed on Apr. 14, 2017.

(51) Int. Cl. A61K 31/7115; A61P 31/18; A61K 31/675; A61P 31/20; A61K 9/00

(52) U.S. Cl. CPC A61K 31/7115; A61P 31/18; A61K 31/706; A61P 31/20; A61K 9/0053; A61K 31/675

(57) ABSTRACT

The present application relates to a method of treating and/or preventing a HBV disease or HIV disease or modulating HBV or HIV replication, comprising administering to a subject in need thereof a compound of Formula I:

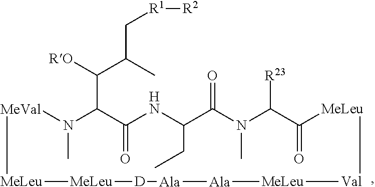

or a pharmaceutically acceptable salt thereof, in combination with a nucleoside analog reverse transcriptase inhibitor or a nucleotide analog reverse transcriptase inhibitor.

### CROSS REFERENCE TO RELATED APPLICATIONS

This application claims priority to, and the benefit of, U.S. Provisional Patent Application No. 62/485,510, filed Apr. 14, 2017, the contents of which are hereby incorporated by reference in their entirety.

### BACKGROUND

The hepatitis B virus (“HBV”) and the human immunodeficiency virus (“HIV”) afflict millions of individuals worldwide and cause a great number of deaths annually. The primary goal of treating chronic hepatitis B is to suppress HBV replication and induce liver disease remission prior to the onset of cirrhosis and hepatocellular carcinoma. None of the approved drugs is completely effective at suppressing HBV replication. Also, available HIV therapies frequently fail due to the development of drug resistance, non-compliance with complicated dosing regimens, pharmacokinetic interactions, toxicity, and/or lack of potency. The limited efficacy of current treatments highlights the need for new therapeutic tools for treating HBV and HIV infections.

3-(hexadecyloxy)propyl hydrogen ((R)-1-(6-amino-9H-purin 9-yl) propan-2-yloxy)methylphosphonate, a lipid conjugate of tenofovir, mimics lysophosphatidylcholine to take advantage of natural lipid uptake pathways and to achieve high intracellular concentrations, thus increasing the effectiveness of tenofovir (“TFV”) against wild-type and mutant HIV and other viruses, such as HBV. 3-(hexadecyloxy)propyl hydrogen ((R)-1-(6-amino-9H-purin 9-yl) propan-2-yloxy)methylphosphonate may be used to treat HIV and/or HBV and inhibit the development of resistance to other antiviral compounds (see WO 2009/094191 and WO 2009/094190).

Cyclosporines are a class of cyclic polypeptides, targeting calcineurin, cyclophilin (“CyP”) isoforms, and P-glycoprotein (“PgP”). Of the cyclosporine compounds, Cyclosporine A (“CsA”) is the most widely used medically. Non-naturally occurring cyclosporines have been prepared, such as cyclosporine analogs modified at amino acid 1 and/or amino acid 3. CyPs provide additional drug targets for hepatitis B treatment. Although cyclosporines can be useful for treating hepatitis B infection, the concomitant effects of immunosuppression limit the utility. Only a few CsA analogs have been proven to show little or reduced immunosuppressive activity and still retain their ability to bind CyPs.

Moreover, the complexity of the viral life cycle necessitates drugs with complementary modes of action, such as a combination of drugs that target the various stages of the HBV or HIV life cycle. Drugs with differing MOAs are theoretically desirable since they have the potential to simultaneously enhance potency and reduce toxicity. However, in practice, most drug combinations are simply additive, or adversely, one or more drugs in the combination may have an antagonistic effect towards the other drug(s), leading to increased toxicity and/or reduced potency. Therefore, there is an unmet need for a combination therapy where the drugs act synergistically in treating HBV and/or HIV infection. The present application addresses that need.

### SUMMARY

The present application relates to a method of treating and/or preventing a hepatitis B virus (“HBV”) disease or a human immunodeficiency virus (“HIV”) disease, comprising administering to a subject in need thereof a compound of Formula I:

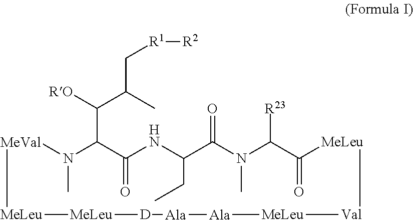

or a pharmaceutically acceptable salt thereof, wherein R′, R′, R², and R²³ are each as defined herein below, in combination with a nucleoside analog reverse transcriptase inhibitor (“NRTI”) or a nucleotide analog reverse transcriptase inhibitor (“NtRTI”).

The present application also relates to a method of treating and/or preventing a HBV disease or a HIV disease, comprising administering to a subject in need thereof a compound of Formula I, or a pharmaceutically acceptable salt thereof, in combination with a compound of Formula II:

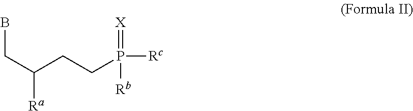

or a pharmaceutically acceptable salt thereof, wherein B, R^{a}, R^{b}, R^{c}, and X are each as defined herein below.

The present application relates to a method of treating and/or preventing a HBV disease or a HIV disease, comprising administering to a subject in need thereof a compound of Formula I, or a pharmaceutically acceptable salt thereof, in combination with tenofovir (also referred to as TFV):

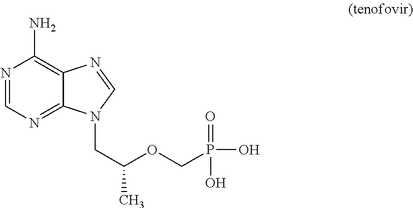 [SMILES: C[C@H](Cn1cnc2c(N)ncnc21)OCP(=O)(O)O]

or a pharmaceutically acceptable salt or prodrug thereof.

The present application relates to a method of treating and/or preventing a HBV disease or a HIV disease, comprising administering to a subject in need thereof a compound of Formula I, or a pharmaceutically acceptable salt thereof, in combination with Compound II:

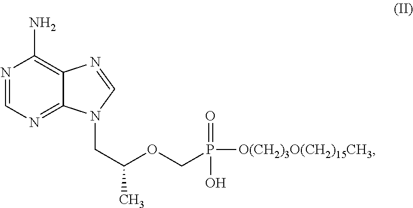

or a pharmaceutically acceptable salt thereof.

The present application relates to a method of treating and/or preventing a HBV disease or a HIV disease, comprising administering to a subject in need thereof a compound of Formula I-A:

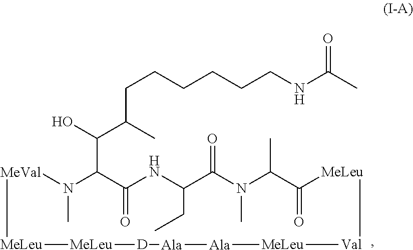

or a pharmaceutically acceptable salt thereof, in combination with a NRTI or a NtRTI.

The present application relates to a method of treating and/or preventing a HBV disease or a HIV disease, comprising administering to a subject in need thereof a compound of Formula I (e.g., Compound I), or a pharmaceutically acceptable salt thereof, in combination with a compound of Formula II, or a pharmaceutically acceptable salt thereof.

The present application relates to a method of treating and/or preventing a HBV disease or a HIV disease, comprising administering to a subject in need thereof a compound of Formula I (e.g., Compound I), or a pharmaceutically acceptable salt thereof, in combination with tenofovir, or a pharmaceutically acceptable salt or prodrug thereof.

The present application relates to a method of treating and/or preventing a HBV disease or a HIV disease, comprising administering to a subject in need thereof a compound of Formula I (e.g., Compound I), or a pharmaceutically acceptable salt thereof, in combination with Compound II, or a pharmaceutically acceptable salt thereof.

The present application also relates to a method of modulating (e.g., inhibiting or decreasing) HBV or HIV replication, comprising administering to a subject in need thereof a compound of Formula I (e.g., Compound I), or a pharmaceutically acceptable salt thereof, in combination with a NRTI or a NtRTI.

The present application also relates to a method of modulating (e.g., inhibiting or decreasing) HBV or HIV replication, comprising administering to a subject in need thereof a compound of Formula I (e.g., Compound I), or a pharmaceutically acceptable salt thereof, in combination with a compound of Formula II, or a pharmaceutically acceptable salt thereof.

The present application also relates to a method of modulating (e.g., inhibiting or decreasing) HBV or HIV replication, comprising administering to a subject in need thereof a compound of Formula I (e.g., Compound I), or a pharmaceutically acceptable salt thereof, in combination with tenofovir, or a pharmaceutically acceptable salt or prodrug thereof.

The present application also relates to a method of modulating (e.g., inhibiting or decreasing) HBV or HIV replication, comprising administering to a subject in need thereof a compound of Formula I (e.g., Compound I), or a pharmaceutically acceptable salt thereof, in combination with Compound II, or a pharmaceutically acceptable salt thereof.

The present application also relates to a kit for the treatment and/or prevention of a HBV disease or a HIV disease, or modulation (e.g., inhibition or decrease) of HBV or HIV replication, comprising a first container comprising a compound of Formula I (e.g., Compound I), or a pharmaceutically acceptable salt thereof, and a second container comprising:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof.

The present application also relates to a compound of Formula I (e.g., Compound I), or a pharmaceutically acceptable salt thereof, for use in treating and/or preventing a HBV disease or a HIV disease, or modulating (e.g., inhibiting or decreasing) HBV or HIV replication in a subject in need thereof, wherein the subject is also administered:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof.

The present application also relates to:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

for use in treating and/or preventing a HBV disease or a HIV disease, or modulating (e.g., inhibiting or decreasing) HBV or HIV replication in a subject in need thereof, wherein the subject is also administered a compound of Formula I (e.g., Compound I), or a pharmaceutically acceptable salt thereof.

The present application also relates to a compound of Formula I (e.g., Compound I), or a pharmaceutically acceptable salt thereof, for use in the manufacture of a medicament for the treatment and/or prevention of a HBV disease or a HIV disease, or modulation (e.g., inhibition or decrease) of HBV or HIV replication in a subject in need thereof, wherein the subject is also administered:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof.

The present application also relates to:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

for use in the manufacture of a medicament for the treatment and/or prevention of a HBV disease or a HIV disease, or modulation (e.g., inhibition or decrease) of HBV or HIV replication in a subject in need thereof, wherein the subject is also administered a compound of Formula I (e.g., Compound I), or a pharmaceutically acceptable salt thereof.

The present application also relates to use of a compound of Formula I (e.g., Compound I), or a pharmaceutically acceptable salt thereof, in the manufacture of a medicament for the treatment and/or prevention of a HBV disease or a HIV disease, or modulation (e.g., inhibition or decrease) of HBV or HIV replication in a subject in need thereof, wherein the subject is also administered:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof.

The present application also relates to use of:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

in the manufacture of a medicament for the treatment and/or prevention of a HBV disease or a HIV disease, or modulation (e.g., inhibition or decrease) of HBV or HIV replication in a subject in need thereof, wherein the subject is also administered a compound of Formula I (e.g., Compound I), or a pharmaceutically acceptable salt thereof.

The present application also relates to a compound of Formula I (e.g., Compound I), or a pharmaceutically acceptable salt thereof, for use in combination with:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

in treating and/or preventing a HBV disease or a HIV disease, or modulating (e.g., inhibiting or decreasing) HBV or HIV replication in a subject in need thereof.

The present application also relates to:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

for use in combination with a compound of Formula I (e.g., Compound I), or a pharmaceutically acceptable salt thereof, in treating and/or preventing a HBV disease or a HIV disease, or modulating (e.g., inhibiting or decreasing) HBV or HIV replication in a subject in need thereof.

The present application also relates to a compound of Formula I (e.g., Compound I), or a pharmaceutically acceptable salt thereof, for use in the manufacture of a medicament for combinational therapy with:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

in the treatment and/or prevention of a HBV disease or a HIV disease, or modulation (e.g., inhibition or decrease) of HBV or HIV replication in a subject in need thereof.

The present application also relates to:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

for use in the manufacture of a medicament for combinational therapy with a compound of Formula I (e.g., Compound I), or a pharmaceutically acceptable salt thereof, in the treatment and/or prevention of a HBV disease or a HIV disease, or modulation (e.g., inhibition or decrease) of HBV or HIV replication in a subject in need thereof.

The present application also relates to use of a compound of Formula I (e.g., Compound I), or a pharmaceutically acceptable salt thereof, in the manufacture of a medicament for combinational therapy with:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

in the treatment and/or prevention of a HBV disease or a HIV disease, or modulation (e.g., inhibition or decrease) of HBV or HIV replication in a subject in need thereof.

The present application also relates to use of:

a NRTI or a NtRTI,

a compound of Formula II, or a pharmaceutically acceptable salt thereof,

tenofovir, or a pharmaceutically acceptable salt or prodrug thereof, or

Compound II, or a pharmaceutically acceptable salt thereof,

in the manufacture of a medicament for combinational therapy with a compound of Formula I (e.g., Compound I), or a pharmaceutically acceptable salt thereof, in the treatment and/or prevention of a HBV disease or a HIV disease, or modulation (e.g., inhibition or decrease) of HBV or HIV replication in a subject in need thereof.

In one embodiment, the NRTI or the NtRTI is a pharmaceutically acceptable salt of Compound II. In one embodiment, the pharmaceutically acceptable salt of Compound II is

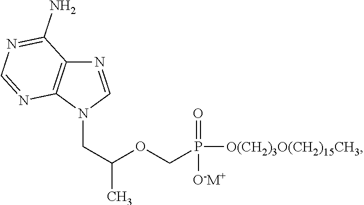

wherein M⁺ is Na⁺, Li⁺, K⁺, Ca²⁺, Mg²⁺, or NR^{d}R^{e}R^{f}R^{g+} and R^{d}, R^{e}, R^{f}, and R^{g} are each independently hydrogen or C₁₋₅ alkyl. For Compound II, when M⁺ is Ca²⁺ or Mg²⁺, two equivalents of the anions are present so as to form neutral molecules.

In one embodiment, the present application relates to treating/treatment of a HBV disease or a HIV disease in a subject in need thereof. In one embodiment, the present application relates to treating/treatment of a HBV disease in a subject in need thereof. In one embodiment, the present application relates to preventing/prevention of a HBV disease or a HIV disease in a subject in need thereof. In one embodiment, the present application relates to preventing/prevention of a HBV disease in a subject in need thereof.

In one aspect, the present disclosure presents a method of treating or preventing a hepatitis B virus (HBV) disease or a human immune deficiency (HIV) disease, comprising administering to a subject in need thereof a compound of Formula I:

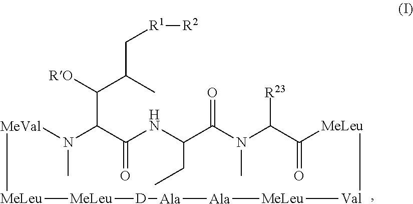

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

in combination with a nucleoside analog reverse transcriptase inhibitor or a nucleotide analog reverse transcriptase inhibitor.

In another aspect, the present disclosure provides a method of modulating HBV or HIV replication in a cell comprising a HBV DNA or HIV DNA, comprising contacting the cell with an effective amount of a compound of Formula I:

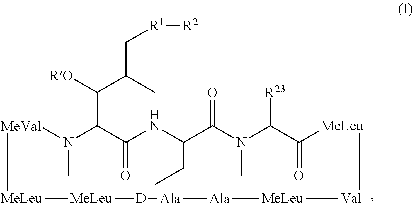

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

in combination with an effective amount of a nucleoside analog reverse transcriptase inhibitor or a nucleotide analog reverse transcriptase inhibitor.

In another aspect, the present disclosure provides a method of modulating HBV or HIV replication, comprising administering to a subject in need thereof a compound of Formula I:

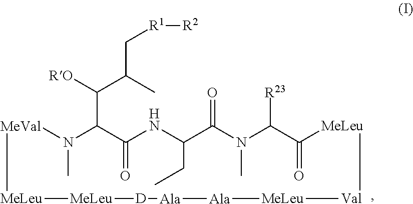

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

in combination with a nucleoside analog reverse transcriptase inhibitor or a nucleotide analog reverse transcriptase inhibitor.

In another aspect, the present disclosure provides a compound of Formula I:

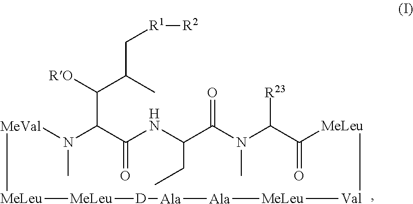

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

for use in treating or preventing a HBV disease or a HIV disease, or modulating HBV or HIV replication in a subject in need thereof, wherein the subject is also administered a nucleoside analog reverse transcriptase inhibitor or a nucleotide analog reverse transcriptase inhibitor.

In another aspect, the present disclosure provides a nucleoside analog reverse transcriptase inhibitor or a nucleotide analog reverse transcriptase inhibitor for use in treating or preventing a HBV disease or a HIV disease, or modulating HBV or HIV replication in a subject in need thereof, wherein the subject is also administered a compound of Formula I:

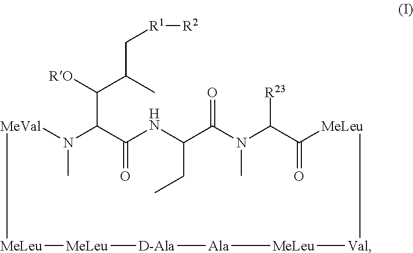

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain.

In another aspect, the present disclosure provides a compound of Formula I:

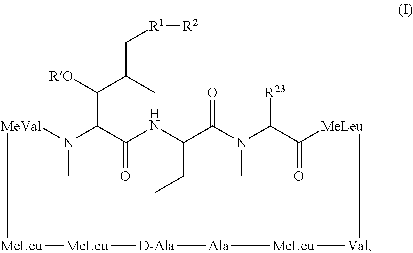

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

for use in the manufacture of a medicament for the treatment or prevention of a HBV disease or a HIV disease, or modulation of HBV or HIV replication in a subject in need thereof, wherein the subject is also administered a nucleoside analog reverse transcriptase inhibitor or a nucleotide analog reverse transcriptase inhibitor.

In another aspect, the present disclosure provides a nucleoside analog reverse transcriptase inhibitor or a nucleotide analog reverse transcriptase inhibitor for use in the manufacture of a medicament for the treatment or prevention of a HBV disease or a HIV disease, or modulation of HBV or HIV replication in a subject in need thereof, wherein the subject is also administered a compound of Formula I:

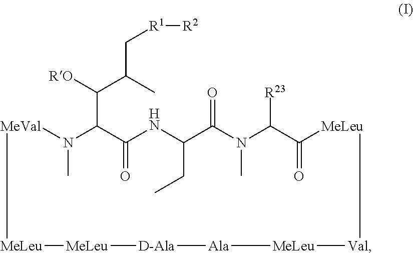

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain.

In another aspect, the present disclosure provides the use of a compound of Formula I:

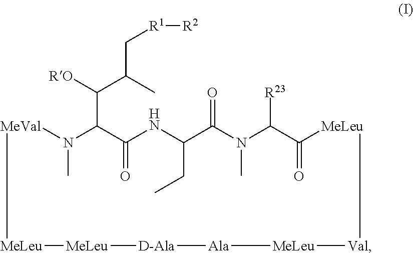

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

in the manufacture of a medicament for the treatment or prevention of a HBV disease or a HIV disease, or modulation of HBV or HIV replication in a subject in need thereof, wherein the subject is also administered a nucleoside analog reverse transcriptase inhibitor or a nucleotide analog reverse transcriptase inhibitor.

In another aspect, the present disclosure provides the use of a nucleoside analog reverse transcriptase inhibitor or a nucleotide analog reverse transcriptase inhibitor in the manufacture of a medicament for the treatment or prevention of a HBV disease or a HIV disease, or modulation of HBV or HIV replication in a subject in need thereof, wherein the subject is also administered a compound of Formula I:

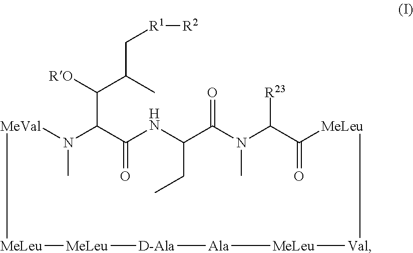

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain.

In another aspect, the present disclosure provides a compound of Formula I:

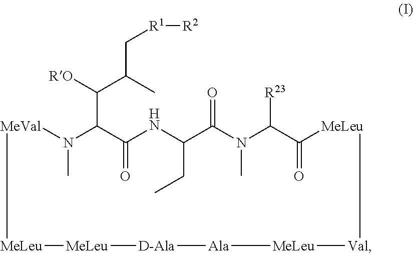

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

for use in combination with a nucleoside analog reverse transcriptase inhibitor or a nucleotide analog reverse transcriptase inhibitor, for treating or preventing a HBV disease or a HIV disease, or modulating HBV or HIV replication in a subject in need thereof.

In another aspect, the present disclosure provides a nucleoside analog reverse transcriptase inhibitor or a nucleotide analog reverse transcriptase inhibitor for use in combination with a compound of Formula I:

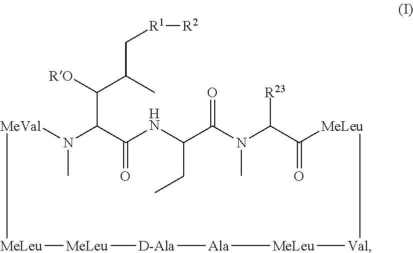

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

for treating or preventing a HBV disease or a HIV disease, or modulating HBV or HIV replication in a subject in need thereof.

In another aspect, the present disclosure provides a compound of Formula I:

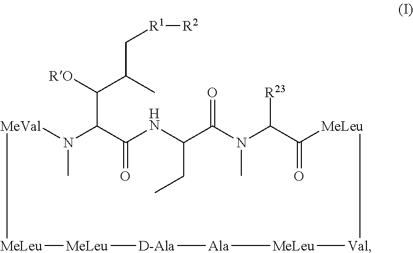

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

for use in the manufacture of a medicament for combinational therapy with a nucleoside analog reverse transcriptase inhibitor or a nucleotide analog reverse transcriptase inhibitor, in the treatment or prevention of a HBV disease or a HIV disease, or modulation of HBV or HIV replication in a subject in need thereof.

In another aspect, the present disclosure provides a nucleoside analog reverse transcriptase inhibitor or a nucleotide analog reverse transcriptase inhibitor for use in the manufacture of a medicament for combinational therapy with a compound of Formula I:

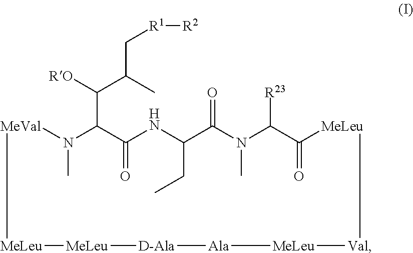

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

in the treatment or prevention of a HBV disease or a HIV disease, or modulation of HBV or HIV replication in a subject in need thereof.

In another aspect, the present disclosure provides the use of a compound of Formula I:

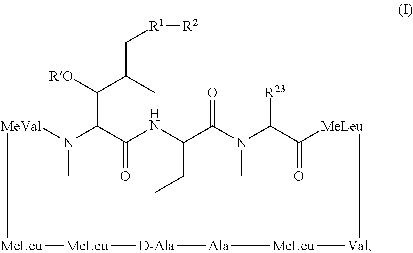

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

in the manufacture of a medicament for combinational therapy with a nucleoside analog reverse transcriptase inhibitor or a nucleotide analog reverse transcriptase inhibitor, for the treatment or prevention of a HBV disease or a HIV disease, or modulation of HBV or HIV replication in a subject in need thereof.

In another aspect, the present disclosure provides the use of a nucleoside analog reverse transcriptase inhibitor or a nucleotide analog reverse transcriptase inhibitor in the manufacture of a medicament for combinational therapy with a compound of Formula I:

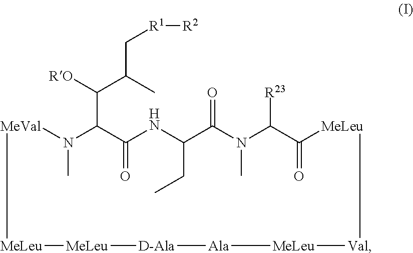

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

for treatment or prevention of a HBV disease or a HIV disease, or modulation of HBV or HIV replication in a subject in need thereof.

In another aspect, the present disclosure provides a kit for the treatment or prevention of a HBV disease or a HIV disease, or modulation of HBV of HIV replication, comprising a first container comprising a compound of Formula I:

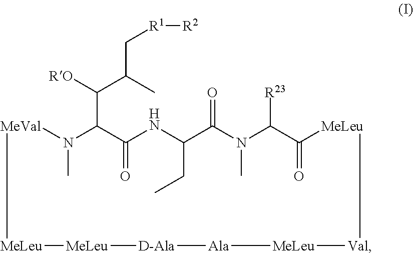

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

and a second container comprising a nucleoside analog reverse transcriptase inhibitor or a nucleotide analog reverse transcriptase inhibitor.

In another aspect, the present disclosure provides a pharmaceutical composition comprising a compound of Formula I:

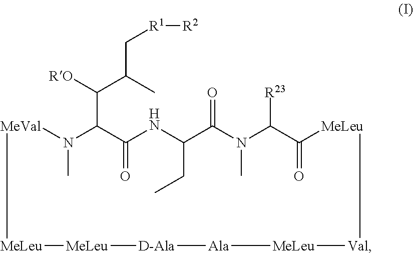

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

and a nucleoside analog reverse transcriptase inhibitor or a nucleotide analog reverse transcriptase inhibitor.

In another aspect, the present disclosure provides a pharmaceutical composition comprising a compound of Formula I:

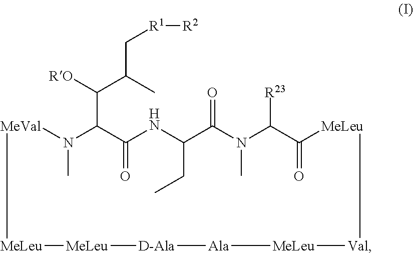

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

and a nucleoside analog reverse transcriptase inhibitor or a nucleotide analog reverse transcriptase inhibitor;

for use in treatment or prevention of HBV disease or HIV disease or modulation of HBV or HIV replication in a subject in need thereof.

In another aspect, the present disclosure provides a method of treating or preventing HBV disease or HIV disease or modulating HBV or HIV replication in a subject in need thereof, comprising administering to the subject in need thereof a pharmaceutical composition comprising a compound of Formula I:

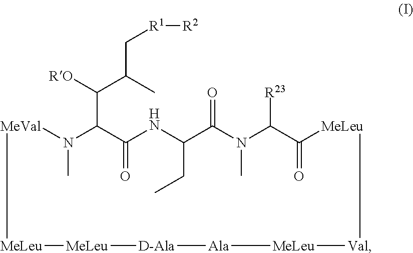

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

and a nucleoside analog reverse transcriptase inhibitor or a nucleotide analog reverse transcriptase inhibitor;

In another aspect, the present disclosure provides the use of a pharmaceutical composition comprising a compound of Formula I:

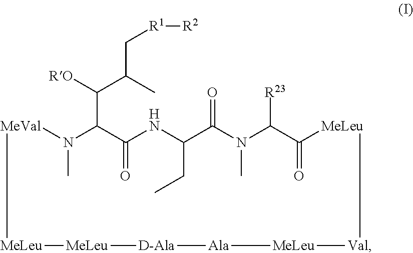

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

and a nucleoside analog reverse transcriptase inhibitor or a nucleotide analog reverse transcriptase inhibitor in the manufacture of a medicament for treating or preventing HBV disease or HIV disease or modulating HBV or HIV replication in a subject in need thereof.

In another aspect, the present disclosure provides a product comprising a compound of Formula I:

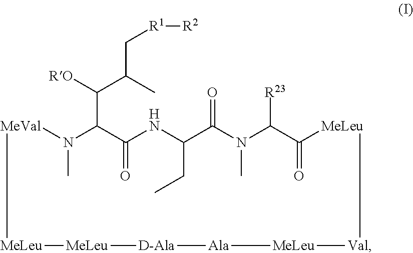

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

and a nucleoside analog reverse transcriptase inhibitor or a nucleotide analog reverse transcriptase inhibitor;

as a combined preparation for simultaneous, separate, or sequential use in the treatment or prevention HBV disease or HIV disease or modulation HBV or HIV replication in a subject in need thereof.

In another aspect, the present disclosure provides a synergistic composition of a nucleoside analog reverse transcriptase inhibitor or a nucleotide analog reverse transcriptase inhibitor and a compound of Formula I:

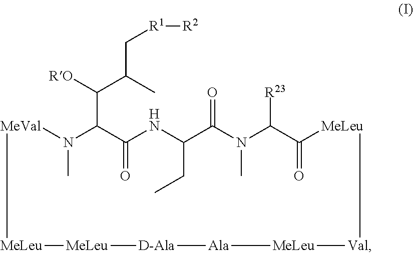

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

in the treatment or prevention of a HBV disease or a HIV disease, or modulation of HBV or HIV replication in a subject in need thereof, wherein the nucleoside analog reverse transcriptase inhibitor or the nucleotide analog reverse transcriptase inhibitor and the compound of Formula I come into contact with each other in the human body (e.g., only in the human body).

In another aspect, the present disclosure provides a method of preparing a composition by bringing a nucleoside analog reverse transcriptase inhibitor or a nucleotide analog reverse transcriptase inhibitor into contact with a compound of Formula I or a pharmaceutically acceptable salt thereof at a locus, wherein Formula I has the structure:

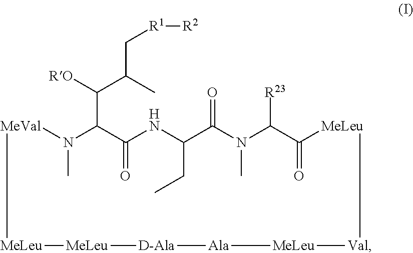

wherein:

R′ is H or acetyl;

R¹ is a saturated or unsaturated straight or branched aliphatic carbon chain from 2 to 15 carbon atoms in length;

R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;

wherein the saturated or unsaturated straight or branched aliphatic carbon chain is optionally substituted with the aromatic group;

wherein the aromatic group is optionally substituted with the saturated or unsaturated straight or branched aliphatic carbon chain; and

R²³ is a saturated or unsaturated straight or branched optionally substituted aliphatic carbon chain;

into contact with each other at a locus.

In some embodiments of any of the above-aspects, in Formula I, R′ is H; 10-R² is

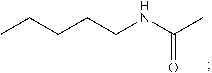

and R²³ is methyl. In some embodiments, the compound of Formula I is a compound of Formula I-A:

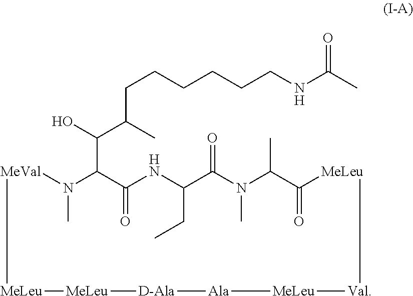

In some embodiments, the compound of Formula I is a D-epimer, wherein the chiral center of the D-epimer is the carbon atom to which R²³ is attached. In some embodiments, the compound of Formula I is Compound I:

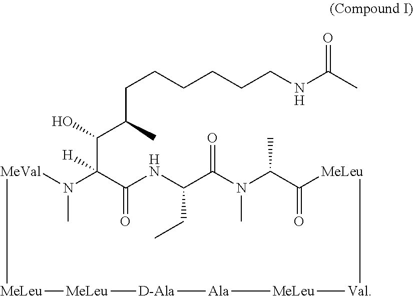

In some embodiments, the compound of Formula I, or a pharmaceutically acceptable salt thereof, and the nucleoside analog reverse transcriptase inhibitor or the nucleotide analog reverse transcriptase inhibitor, are administered simultaneously. In some embodiments, the compound of Formula I, or a pharmaceutically acceptable salt thereof, and the nucleoside analog reverse transcriptase inhibitor or the nucleotide analog reverse transcriptase inhibitor, are administered sequentially.

In some embodiments, the compound of Formula I, or a pharmaceutically acceptable salt thereof, is administered prior to the nucleoside analog reverse transcriptase inhibitor or the nucleotide analog reverse transcriptase inhibitor. In some embodiments, the nucleoside analog reverse transcriptase inhibitor or the nucleotide analog reverse transcriptase inhibitor is administered prior to the compound of Formula I, or a pharmaceutically acceptable salt thereof. In some embodiments, the compound of Formula I, or a pharmaceutically acceptable salt thereof, and the nucleoside analog reverse transcriptase inhibitor or the nucleotide analog reverse transcriptase inhibitor, are administered in alternation.

In some embodiments, the compound of Formula I, or a pharmaceutically acceptable salt thereof, and the nucleoside analog reverse transcriptase inhibitor or the nucleotide analog reverse transcriptase inhibitor, are comprised in the same dosage unit form. In some embodiments, the compound of Formula I, or a pharmaceutically acceptable salt thereof, is administered orally. In some embodiments, the nucleoside analog reverse transcriptase inhibitor or the nucleotide analog reverse transcriptase inhibitor is administered orally. In some embodiments, the compound of Formula I, or a pharmaceutically acceptable salt thereof, is administered in a therapeutically effective amount.

In some embodiments, the compound of Formula I, or a pharmaceutically acceptable salt thereof, is administered in an amount from about 1 mg per day to about 1,000 mg per day. In some embodiments, the nucleoside analog reverse transcriptase inhibitor or the nucleotide analog reverse transcriptase inhibitor is administered in a therapeutically effective amount.

In some embodiments, the nucleoside analog reverse transcriptase inhibitor or the nucleotide analog reverse transcriptase inhibitor is administered in an amount from about 5 mg per day to about 400 mg per day. In some embodiments, the compound of Formula I, or a pharmaceutically acceptable salt thereof, is administered once, twice, or at least three times daily. In some embodiments, the compound of Formula I, or a pharmaceutically acceptable salt thereof, is administered once daily, once every two days, or once every three days. In some embodiments, the compound of Formula I, or a pharmaceutically acceptable salt thereof, is administered once weekly. In some embodiments, the compound of Formula I, or a pharmaceutically acceptable salt thereof, is administered for 1, 2, 3, 4, 5, 6, or 7 days a week. In some embodiments, the nucleoside analog reverse transcriptase inhibitor or the nucleotide analog reverse transcriptase inhibitor is administered once, twice, or at least three times daily. In some embodiments, the nucleoside analog reverse transcriptase inhibitor or the nucleotide analog reverse transcriptase inhibitor is administered once daily, once every two days, or once every three days. In some embodiments, the nucleoside analog reverse transcriptase inhibitor or a nucleotide analog reverse transcriptase inhibitor is administered once weekly. In some embodiments, the nucleoside analog reverse transcriptase inhibitor or the nucleotide analog reverse transcriptase inhibitor is administered for 1, 2, 3, 4, 5, 6, or 7 days a week.

In some embodiments, the compound of Formula I, or a pharmaceutically acceptable salt thereof, and the nucleoside analog reverse transcriptase inhibitor or the nucleotide analog reverse transcriptase inhibitor, are administered with the same dosing frequency. In some embodiments, the compound of Formula I, or a pharmaceutically acceptable salt thereof, is administered for at least a month, for at least three months, for at least six months, for at least five years, for at least ten years, or for the duration of the subject's life. In some embodiments, the nucleoside analog reverse transcriptase inhibitor or the nucleotide analog reverse transcriptase inhibitor is administered for at least a month, for at least three months, for at least six months, for at least five years, for at least ten years, or for the duration of the subject's life.

In some embodiments, the compound of Formula I, or a pharmaceutically acceptable salt thereof, and the nucleoside analog reverse transcriptase inhibitor or the nucleotide analog reverse transcriptase inhibitor, are administered with the same treatment duration. In some embodiments, the subject is a human being.

In some embodiments of any of the above aspects, the nucleoside analog reverse transcriptase inhibitor or the nucleotide analog reverse transcriptase inhibitor is a compound of Formula II:

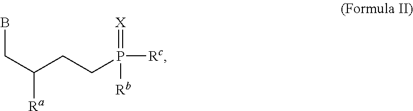

or a pharmaceutically acceptable salt thereof, wherein:

B is a purine or pyrimidine base;

R^{a} is H, methyl, ethyl, —CH₂OH, —CH₂—CH₂OH, —CH(OH)—CH₃, or C₁₋₆ haloalkyl;

R^{b} is fluoro, hydroxy, —OR^{2a}, —BH₃, C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, or —NR′H;

R^{2a} is C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, —P(═O)(OH)₂, or —P(═O)(OH)OP(═O)(OH)₂;

R′ is C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, or C₆₋₁₀ aryl;

R^{c} is —OH or —O(CH₂)ₘO(CH₂)ₙCH₃, wherein m is from 2 to 5 and n is from 11 to 21; and

X is selenium, sulphur, or oxygen.

In some embodiments, the nucleoside analog reverse transcriptase inhibitor or the nucleotide analog reverse transcriptase inhibitor is tenofovir:

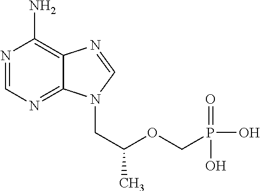 [SMILES: C[C@H](Cn1cnc2c(N)ncnc21)OCP(=O)(O)O]

or a pharmaceutically acceptable salt or prodrug thereof.

In some embodiments, the nucleoside analog reverse transcriptase inhibitor or the nucleotide analog reverse transcriptase inhibitor is a compound of Formula II-A:

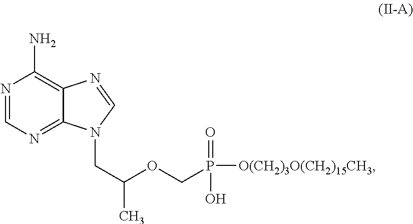

or a pharmaceutically acceptable salt thereof.

In some embodiments, the compound of Formula II-A is Compound II:

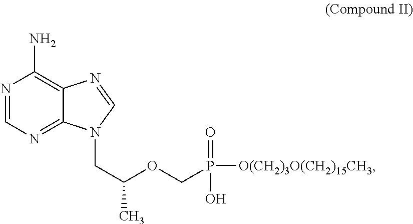

or a pharmaceutically acceptable salt thereof.

In some embodiments, the pharmaceutically acceptable salt of the compound of Formula II-A is:

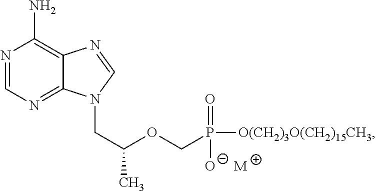

wherein M⁺ is Na⁺, K⁺, Ca²⁺, Mg²⁺, or NRₐR_{b}R_{c}R_{d}⁺ and Rₐ, R_{b}, R_{c}, and R_{d} are each independently hydrogen or C₁₋₅ alkyl. In some embodiments, M⁺ is K⁺.

In some embodiments, the effective amounts of the compound of Formula I, or a pharmaceutically acceptable salt thereof, and the nucleoside analog reverse transcriptase inhibitor or the nucleotide analog reverse transcriptase inhibitor, are each an amount sufficient to inhibit or decrease HBV replication by at least 50%. In some embodiments, the effective amounts of the compound of Formula I, or a pharmaceutically acceptable salt thereof, and the nucleoside analog reverse transcriptase inhibitor or the nucleotide analog reverse transcriptase inhibitor, are each an amount sufficient to inhibit or decrease HBV replication by at least 70%. In some embodiments, the effective amounts of the compound of Formula I, or a pharmaceutically acceptable salt thereof, and the nucleoside analog reverse transcriptase inhibitor or the nucleotide analog reverse transcriptase inhibitor, are each an amount sufficient to inhibit or decrease HBV replication by at least 90%.

Unless otherwise defined, all technical and scientific terms used herein have the same meaning as commonly understood by one of ordinary skill in the art to which this application belongs. In the case of conflict, the present specification, including definitions, will control. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art to the present application. In addition, the materials, methods, and examples are illustrative only and are not intended to be limiting.

Other features and advantages of the present application will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows the inhibition of intracellular HBV DNA replication in AD38 cells at various concentrations of Compound II, as a function of Compound I concentration.

FIG. 1B shows the inhibition of intracellular HBV DNA replication in AD38 cells at various concentrations of Compound I, as a function of Compound II concentration.

FIG. 2A shows the inhibition of intracellular HBV DNA replication in DES19 cells at various concentrations of Compound II, as a function of Compound I concentration.

FIG. 2B shows the inhibition of intracellular HBV DNA replication in DES19 cells at various concentrations of Compound I, as a function of Compound II concentration.

FIG. 3A shows the inhibition of intracellular HBV DNA replication in DE19 cells at various concentrations of Compound II, as a function of Compound I concentration.

FIG. 3B shows the inhibition of intracellular HBV DNA replication in DE19 cells at various concentrations of Compound I, as a function of Compound II concentration.

FIG. 4A is a synergy plot showing the log volumes for the combination of Compound I (0-320 nM) and Compound II (0-640 nM) in DE19 cells. The flat plane represents additive effects. Upward peaks and downward peaks, if any, represent synergistic and antagonistic effects, respectively.

FIG. 4B is a synergy plot showing the log volumes for the combination of Compound I (0-320 nM) and Compound II (0-640 nM) in DES19 cells. The flat plane represents additive effects. Upward peaks and downward peaks, if any, represent synergistic and antagonistic effects, respectively.

FIG. 5 is a synergy plot showing synergy scores for the combinations of Compound I (0-320 nM) and Compound II (0-640 nM) in AD38 cells. The flat plane represents additive effects. Upward peaks and downward peaks, if any, represent synergistic and antagonistic effects, respectively.

FIG. 6 shows HBV DNA copies per mg liver after treatment with vehicle, Compound I and/or Compound II.

FIG. 7 shows serum HBsAg levels after treatment with vehicle, Compound I and/or Compound II.

FIG. 8, shows a plots of liver HBsAg after treatment with vehicle, Compound I and/or Compound II.

FIG. 9 shows a plot of serum HBV DNA after treatment with vehicle, Compound I and/or Compound II.

FIG. 10 shows a plot of serum HBeAg levels after treatment with vehicle, Compound I and/or Compound II.

FIG. 11 shows a plot of compound I levels in serum as a function of dose and in combination with compound II.

### DETAILED DESCRIPTION

The present application relates to a method of treating and/or preventing a hepatitis B virus (HBV) disease or a human immunodeficiency virus (“HIV”) disease, or modulating (e.g., inhibiting or decreasing) HBV or HIV replication, comprising administering to a subject in need thereof a compound of Formula I, or a pharmaceutically acceptable salt thereof, in combination with a nucleoside analog reverse transcriptase inhibitor (“NRTI”) or a nucleotide analog reverse transcriptase inhibitor (“NtRTI”), wherein the compound of Formula I is shown as follows:

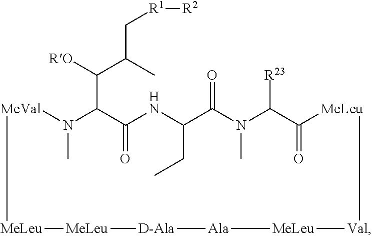

### Formula I

or a pharmaceutically acceptable salt thereof, wherein:

R′ is H or acetyl;

R¹ is a saturated or unsaturated straight or branched aliphatic carbon chain from 2 to 15 carbon atoms in length;

R² is selected from the group consisting of:
H;
an unsubstituted, N-substituted, or N,N-disubstituted amide;
a N-substituted or unsubstituted acyl protected amine;
a carboxylic acid;
a N-substituted or unsubstituted amine;
a nitrile;
an ester;
a ketone;
a hydroxy, dihydroxy, trihydroxy, or polyhydroxy alkyl;
a substituted or unsubstituted aryl;
a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo;
an aromatic group containing a substituent selected from the group consisting of halides, esters, and nitro; and
wherein the saturated or unsaturated straight or branched aliphatic carbon chain is optionally substituted with the aromatic group;
wherein the aromatic group is optionally substituted with the saturated or unsaturated straight or branched aliphatic carbon chain; and

R²³ is a saturated or unsaturated straight or branched optionally substituted aliphatic carbon chain.

In one embodiment, R¹-R² is selected from the group consisting of:

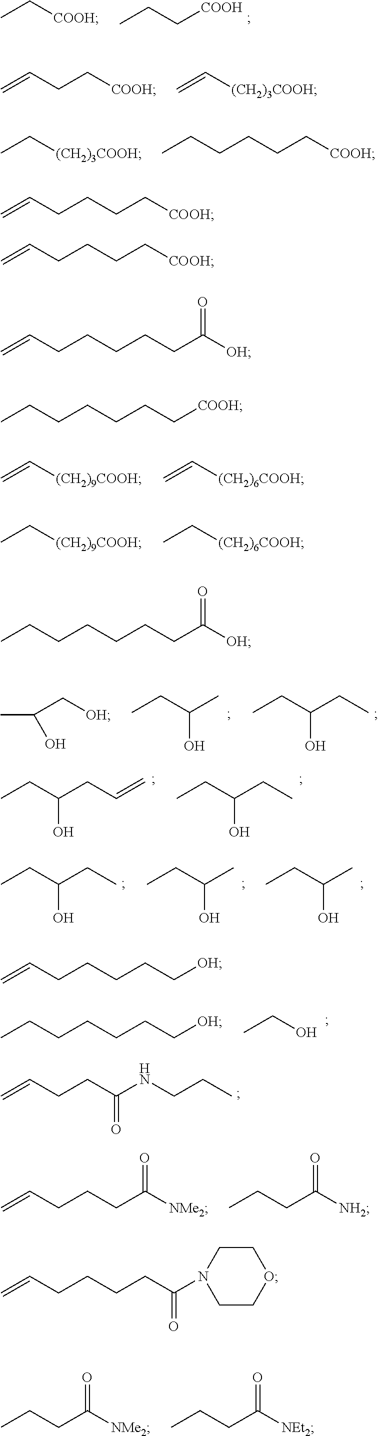

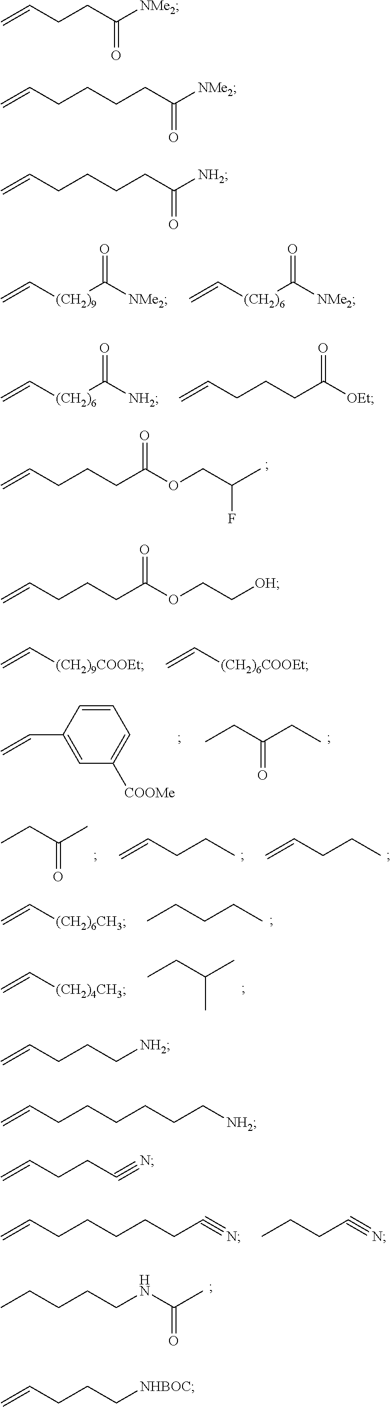

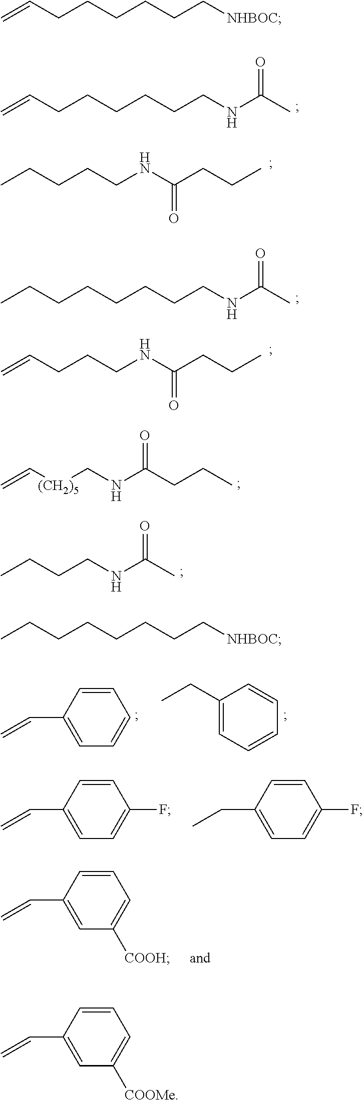

In another embodiment, R² is selected from the group consisting of:

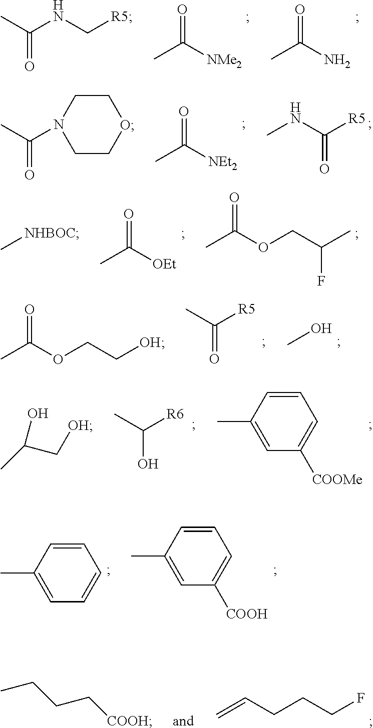

wherein:

R⁵ is a saturated or unsaturated straight or branched aliphatic carbon chain between 1 and 10 carbons in length; and

R⁶ is a monohydroxylated, dihydroxylated, trihydroxylated, or polyhydroxylated saturated or unsaturated straight or branched aliphatic carbon chain between 1 and 10 carbons in length.

In one embodiment, R¹-R² comprises a saturated or unsaturated straight or branched aliphatic carbon chain of between 2 and 5 carbons optionally substituted with a substituent selected from the group consisting of ketones, hydroxyls, nitriles, halogens, oxo, carboxylic acids, esters, and 1,3-dioxolanes.

In one embodiment, R²³ is selected from the group consisting of:
—CH₃
—CH₂CH₃
—CH₂CHCH₂
—CH₂CH₂CH₂I
—(CH₂)₃CH₂I
—(CH₂)₃N⁺(CH₃)₃
—CH₂CCH
—CH₂CO₂(t-Bu)
—CH₂Ph
—CH₂OH
—CH(OH)CH₃
—CH(OH)(t-Bu)
—CH(OH)Ph
—COOH
—SCH₃ and
—S(p-Tol).

In one embodiment, R²³ comprises an optionally substituted alkyl, including optionally substituted C₁-C₃ alkyl. Said alkyl may be substituted with amino and may comprise a C₁-C₃-Ala wherein said compound comprises the D-epimer. In one embodiment, R²³ can be MeAla.

In one embodiment, of the above Formula I,

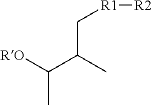

is selected from the group consisting of

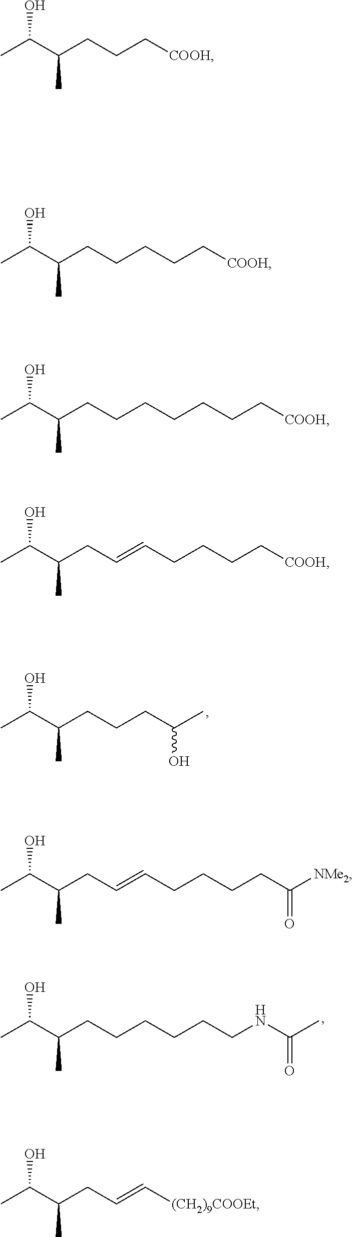

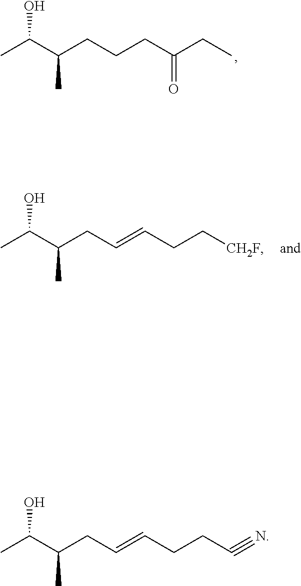

In one embodiment, R²³ is a straight or branched aliphatic carbon chain of 1 to 6, 1 to 5, 1 to 4, 1 to 3 or 2 carbons in length.

Particularly, compounds of Formula I include the compounds wherein R′ is H, R¹ is an alkyl or alkenyl between 2 and 15 carbons (e.g., between 2 and 12 carbons, between 2 and 10 carbons, between 2 and 9 carbons, between 2 and 8 carbons, between 2 and 7 carbons, between 2 and 6 carbons, or between 2 and 6 carbons) in length, and R² is selected from:

a carboxylic acid comprising a carboxyl group;

a N-substituted of N,N-disubstituted amide, wherein the substituents are independently selected from an alkyl between 1 and 7 carbons in length and a heterocyclic ring comprising 1-3 heteroatoms selected from O, N and S;

an ester of between 1 and 7 carbons in length;

a monohydroxylated or dihydroxylated alkyl of between 1 and 7 carbons in length;

a N-substituted or unsubstituted acyl protected amine of between 1 and 7 carbons in length;

a nitrile;

a ketone, wherein the carbonyl group of the ketone is connected to R¹ and an alkyl or alkenyl chain between 1 and 7 carbons in length;

phenyl, optionally substituted with one or more substituents independently selected from nitrogen dioxide, a fluorine, an amine, an ester, and a carboxyl group.

In one embodiment, a compound of Formula I is a compound of Formula I-A:

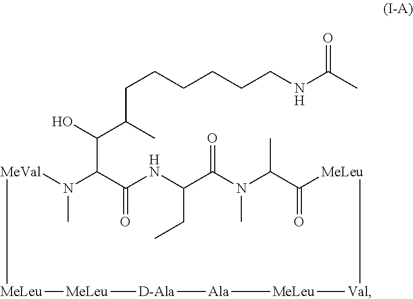

In one embodiment, a compound of Formula I (e.g., Compound I) is a D-epimer, wherein the chiral center of the D-epimer is the carbon atom to which R²³ is attached. In one embodiment, a compound of Formula I (e.g., Compound I) is an L-epimer, wherein the chiral center of the L-epimer is the carbon atom to which R²³ is attached. In one embodiment, a compound of Formula I (e.g., Compound I) is a mixture of D-epimer and L-epimer, wherein the chiral center of the D-epimer and the L-epimer is the carbon atom to which R²³ is attached.

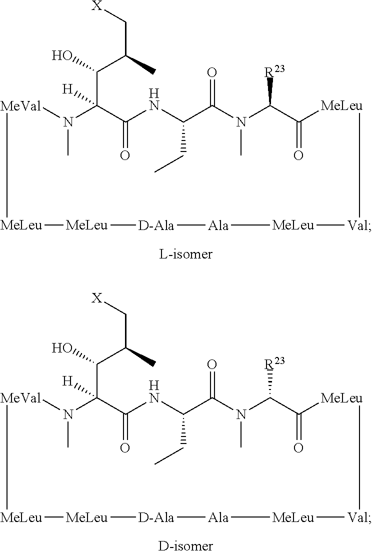

For example, in some embodiments, the compound of Formula I is:

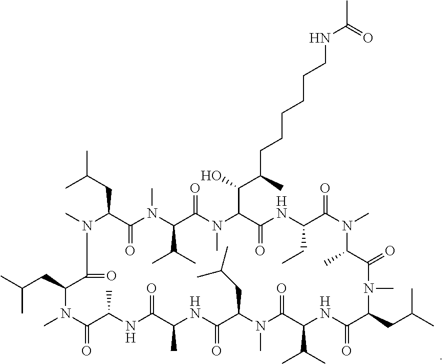 [SMILES: CC[C@@H]1NC(=O)C([C@H](O)[C@H](C)CCCCCCNC(C)=O)N(C)C(=O)[C@@H](C(C)C)N(C)C(=O)[C@H](CC(C)C)N(C)C(=O)[C@H](CC(C)C)N(C)C(=O)[C@H](C)NC(=O)[C@H](C)NC(=O)[C@@H](CC(C)C)N(C)C(=O)[C@H](C(C)C)NC(=O)[C@H](CC(C)C)N(C)C(=O)[C@H](C)N(C)C1=O]

In one embodiment, the NRTI or NtRTI is a compound of Formula II:

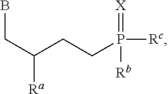

### Formula II

or a pharmaceutically acceptable salt thereof, wherein:

B is a purine or pyrimidine base;

R^{a} is H, methyl, ethyl, —CH₂OH, —CH₂—CH₂OH, —CH(OH)—CH₃, or C₁₋₆ haloalkyl;

R^{b} is fluoro, hydroxy, —OR²a, —BH₃, C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, or —NR′H;

R^{2a} is C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, —P(═O)(OH)₂, or —P(═O)(OH)OP(═O)(OH)₂;

R′ is C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, or C₆₋₁₀ aryl;

R^{c} is —O(CH₂)ₘO(CH₂)ₙCH₃, wherein m is from 2 to 5 and n is from 11 to 21; and

X is selenium, sulphur, or oxygen.

In one embodiment, the NtRTI or the NRTI is a compound of Formula II, wherein B is a purine base.

In one embodiment, the NtRTI or the NRTI is a compound of Formula II, wherein B is a pyrimidine base.

In one embodiment, the NtRTI is tenofovir:

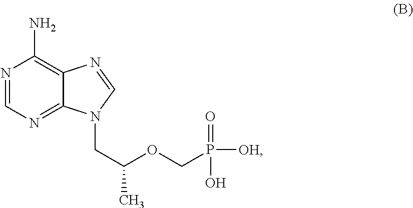 [SMILES: C[C@H](Cn1cnc2c(N)ncnc21)OCP(=O)(O)O]

or a pharmaceutically acceptable salt or prodrug thereof.

In one embodiment, the NtRTI is Compound II:

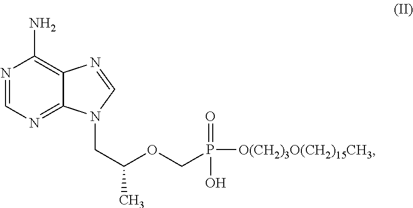

or a pharmaceutically acceptable salt thereof.

In one embodiment, the present application relates to a method of treating and/or preventing a HBV disease or a HIV disease, or modulating (e.g., inhibiting or decreasing) HBV or HIV replication, comprising administering to a subject in need thereof. Compound I, or a pharmaceutically acceptable salt thereof, in combination with tenofovir, or a pharmaceutically acceptable salt or prodrug thereof.

In one embodiment, the present application relates to a method of treating and/or preventing a HBV disease or a HIV disease, or modulating (e.g., inhibiting or decreasing) HBV or HIV replication, comprising administering to a subject in need thereof. Compound I, or a pharmaceutically acceptable salt thereof, in combination with Compound II, or a pharmaceutically acceptable salt thereof.

In one embodiment, the present application relates to a method of treating and/or preventing a HBV disease, or modulating (e.g., inhibiting or decreasing) HBV replication. In one embodiment, the present application relates to a method of treating a HBV disease. In one embodiment, the present application relates to a method of preventing a HBV disease.

In one embodiment, the present application relates to a method of treating and/or preventing a HIV disease, or modulating (e.g., inhibiting or decreasing) HIV replication. In one embodiment, the present application relates to a method of treating a HIV disease. In one embodiment, the present application relates to a method of preventing a HIV disease.

The present application relates to a compound of the present application (e.g., a compound of Formula I (e.g., Compound I)) for use in treating and/or preventing, or for use in the manufacture of a medicament for the treatment and/or prevention of, a HBV disease or a HIV disease, or for use in modulating (e.g., inhibiting or decreasing), or for use in the manufacture of a medicament for the modulation (e.g., inhibition or decrease) of, HBV or HIV replication, in a subject in need thereof, wherein the subject is also administered a NtRTI or a NRTI (e.g., a compound of Formula II, tenofovir, or Compound II).

The present application relates to use of a compound of the present application (e.g., a compound of Formula I (e.g., Compound I)) in the manufacture of a medicament for the treatment and/or prevention of a HBV disease or a HIV disease, or the modulation (e.g., inhibition or decrease) of HBV or HIV replication, in a subject in need thereof, wherein the subject is also administered a NtRTI or a NRTI (e.g., a compound of Formula II, tenofovir, or Compound II).

The present application relates to a NtRTI or a NRTI (e.g., a compound of Formula II, tenofovir, or Compound II) for use in treating and/or preventing, or for use in the manufacture of a medicament for the treatment and/or prevention of, a HBV disease or a HIV disease, or for use in modulating (e.g., inhibiting or decreasing), or for use in the manufacture of a medicament for the modulation (e.g., inhibition or decrease) of, HBV or HIV replication, in a subject in need thereof, wherein the subject is also administered a compound of the present application (e.g., a compound of Formula I (e.g., Compound I)).

The present application relates to use of a NtRTI or a NRTI (e.g., a compound of Formula II, tenofovir, or Compound II) in the manufacture of a medicament for the treatment and/or prevention of a HBV disease or a HIV disease, or the modulation (e.g., inhibition or decrease) of HBV or HIV replication, in a subject in need thereof, wherein the subject is also administered a compound of the present application (e.g., a compound of Formula I (e.g., Compound I)).

The present application relates to a compound of the present application (e.g., a compound of Formula I (e.g., Compound I)) for use in combination with, or for use in the manufacture of a medicament for combinational therapy with, a NtRTI or a NRTI (e.g., a compound of Formula II, tenofovir, or Compound II), in treating and/or preventing a HBV disease or a HIV disease, or modulating (e.g., inhibiting or decreasing) HBV or HIV replication, in a subject in need thereof.

The present application relates to use of a compound of the present application (e.g., a compound of Formula I (e.g., Compound I)) in the manufacture of a medicament for combinational therapy with a NtRTI or a NRTI (e.g., a compound of Formula II, tenofovir, or Compound II) in treating and/or preventing a HBV disease or a HIV disease, or modulating (e.g., inhibiting or decreasing) HBV or HIV replication, in a subject in need thereof.

The present application relates to a NtRTI or a NRTI (e.g., a compound of Formula II, tenofovir, or Compound II) for use in combination with, or for use in the manufacture of a medicament for combinational therapy with, a compound of the present application (e.g., a compound of Formula I (e.g., Compound I)), in treating and/or preventing a HBV disease or a HIV disease, or modulating (e.g., inhibiting or decreasing) HBV or HIV replication in, a subject in need thereof.

The present application relates to use of a NtRTI or a NRTI (e.g., a compound of Formula II, tenofovir, or Compound II) in the manufacture of a medicament for combinational therapy with a compound of the present application (e.g., a compound of Formula I (e.g., Compound I)) in treating and/or preventing a HBV disease or a HIV disease, or modulating (e.g., inhibiting or decreasing) HBV or HIV replication, in a subject in need thereof.

The present application also relates to a kit for the treatment and/or prevention of a HBV disease or a HIV disease, or modulation (e.g., inhibition or decrease) of HBV or HIV replication, comprising a first container comprising a compound of Formula I (e.g., Compound I), or a pharmaceutically acceptable salt thereof, and a second container comprising a NRTI or a NtRTI (e.g., a compound of Formula I, Compound I, a NtRTI or a NRTI, a compound of Formula II, tenofovir, or Compound II). In one embodiment, the present application also relates to a kit for the treatment and/or prevention of a HBV disease or a HIV disease, or modulation (e.g., inhibition or decrease) of HBV or HIV replication, comprising a first container comprising Compound I, and a second container comprising Compound II.

In one embodiment, the compound is Compound I, or a pharmaceutically acceptable salt thereof, and the NtRTI or NRTI is tenofovir, or a pharmaceutically acceptable salt or prodrug thereof. In one embodiment, the compound is Compound I, or a pharmaceutically acceptable salt thereof, and the NtRTI or NRTI is Compound II, or a pharmaceutically acceptable salt thereof.

In one embodiment, the effective amount of a compound of the present application (e.g., a compound of Formula I, Compound I, a NtRTI or a NRTI, a compound of Formula II, tenofovir, or Compound II or a combination thereof) is an amount sufficient to modulate (e.g., inhibit or decrease) HBV or HIV replication by at least 50%. In a further embodiment, the effective amount of a compound of the present application (e.g., a compound of Formula I, Compound I, a NtRTI or a NRTI, a compound of Formula II, tenofovir, or Compound II or a combination thereof) is an amount sufficient to modulate (e.g., inhibit or decrease) HBV or HIV replication by at least 70%. In one embodiment, the effective amount of a compound of the present application (e.g., a compound of Formula I, Compound I, a NtRTI or a NRTI, a compound of Formula II, tenofovir, or Compound II or a combination thereof) is an amount sufficient to modulate (e.g., inhibit or decrease) HBV or HIV replication by at least 90%.

The present application also relates to a method of modulating (e.g., inhibiting or decreasing) HBV or HIV replication in a cell comprising a HBV DNA or a HIV DNA, comprising contacting the cell with an effective amount of a compound of Formula I (e.g., Compound I), or a pharmaceutically acceptable salt thereof, and a NtRTI or a NRTI (e.g., a compound of Formula II, tenofovir, or Compound II). The present application also relates to a method of modulating (e.g., inhibiting or decreasing) HBV or HIV replication in a cell comprising a HBV DNA or a HIV DNA, comprising contacting the cell with an effective amount of Compound I, or a pharmaceutically acceptable salt thereof, and an effective amount of tenofovir, or a pharmaceutically acceptable prodrug or salt thereof. The present application also relates to a method of modulating (e.g., inhibiting or decreasing) HBV or HIV replication in a cell comprising a HBV DNA or a HIV DNA, comprising contacting the cell with an effective amount of Compound I, or a pharmaceutically acceptable salt thereof, and an effective amount of Compound II, or a pharmaceutically acceptable salt thereof.

In one embodiment, the contacting is performed in vitro or ex vivo. In one embodiment, the contacting is performed in vivo, for example, by administering a compound of Formula I (e.g., Compound I), or a pharmaceutically acceptable salt thereof and a NtRTI or a NRTI (e.g., a compound of Formula II, tenofovir, or Compound II), to a subject harboring a cell comprising a HBV DNA or a HIV DNA. In one embodiment, the contacting is performed in vivo, for example, by administering a compound of Formula I (e.g., Compound I), or a pharmaceutically acceptable salt thereof and tenofovir, or a pharmaceutically acceptable salt or prodrug thereof, to a subject harboring a cell comprising a HBV DNA or a HIV DNA. In one embodiment, the contacting is performed in vivo, for example, by administering a compound of Formula I (e.g., Compound I), or a pharmaceutically acceptable salt thereof and Compound II, or a pharmaceutically acceptable salt thereof, to a subject harboring a cell comprising a HBV DNA or a HIV DNA.

In one embodiment, the NRTI is selected from zidovudine, didanosine, zalcitabine, stavudine, lamivudine, abacavir, emtricitabine, entecavir, and telbivudine, and a combination thereof.

In one embodiment, the NtRTI is selected from tenofovir and adefovir, and a combination and prodrugs thereof.

In one embodiment, the NtRTI is a compound of Formula II, wherein B is purine. In another embodiment, the NtRTI is tenofovir or a pharmaceutically acceptable salt or prodrug thereof. In a further embodiment, the NtRTI is a prodrug of tenofovir. In a further embodiment, the NtRTI is Compound II or a pharmaceutically acceptable salt thereof.

In one embodiment the pharmaceutically acceptable salt of Formula II (e.g., Compound II) is

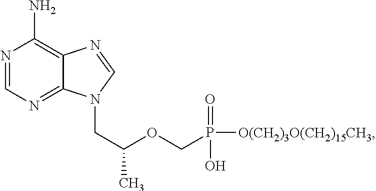

wherein M⁺ is Na⁺, Li⁺, K⁺, Ca²⁺, Mg²⁺, or NR^{d}R^{e}R^{f}R^{g+} and R^{d}, R^{e}, R^{f}, and R^{g} are each independently hydrogen or C₁₋₅ alkyl. In one embodiment, M⁺ is Li⁺, K⁺, Ca²⁺, Mg²⁺, or NR^{d}R^{e}R^{f}R^{g+} and R^{d}, R^{e}, and R^{f} are each independently hydrogen or C₁₋₅ alkyl. In a further embodiment, M⁺ is Na⁺, Li⁺, K⁺, Ca²⁺, Mg²⁺, or NH₄⁺. In a further embodiment, M⁺ is Li⁺, K⁺, Ca²⁺, Mg²⁺, or NH₄⁺. In one embodiment, M⁺ is Na⁺, Li⁺, K⁺, or NH₄⁺. In one embodiment, M⁺ is Li⁺ or NH₄⁺. In one embodiment, M⁺ is K⁺. For Compound II, when M⁺ is Ca²⁺ or Mg²⁺, two equivalents of the anions are present so as to form neutral molecules.

In one embodiment, a compound of Formula I (e.g., Compound I), or a pharmaceutically acceptable salt thereof, and a NtRTI or a NRTI (e.g., a compound of Formula II, tenofovir, or Compound II), are administered simultaneously (i.e., for simultaneous administration). In another embodiment, a compound of Formula I (e.g., Compound I), or a pharmaceutically acceptable salt thereof, and a NtRTI or a NRTI (e.g., a compound of Formula II, tenofovir, or Compound II), are administered sequentially (i.e., for sequential administration). In a further embodiment, a compound of Formula I (e.g., Compound I), or a pharmaceutically acceptable salt thereof, is administered (i.e., for administration) prior to a NtRTI or a NRTI (e.g., a compound of Formula II, tenofovir, or Compound II). In another embodiment, a NtRTI or a NRTI (e.g., a compound of Formula II, tenofovir, or Compound II) is administered (i.e., for administration) prior to a compound of Formula I (e.g., Compound I), or a pharmaceutically acceptable salt thereof. In another embodiment, a compound of Formula I (e.g., Compound I), or a pharmaceutically acceptable salt thereof, and a NtRTI or a NRTI (e.g., a compound of Formula II, tenofovir, or Compound II), are administered in alternation (i.e., for alternate administration).

In some embodiments, the compound of Formula I and the NtRTI or a NRTI are administered in temporal proximity (e.g., the compound of Formula I and the NtRTI or a NRTI can be administered simultaneously). Accordingly, the present disclosure provides a method of treating or preventing a HBV disease or HIV comprising administering a compound of Formula I and an NtRTI or a NRTI in temporal proximity.

In one embodiment, a compound of Formula I (e.g., Compound I), or a pharmaceutically acceptable salt thereof, and a NtRTI or a NRTI (e.g., a compound of Formula II, tenofovir, or Compound II) are comprised in the same dosage unit form. In one embodiment, the dosage unit is in the form of a pill, capsule, or tablet. In one embodiment, the dosage unit is in the form of a suspension, solution, emulsion, mouthwash, or elixir.

In one embodiment, a compound of Formula I (e.g., Compound I), or a pharmaceutically acceptable salt thereof, and a NtRTI or a NRTI (e.g., a compound of Formula II, tenofovir, or Compound II), are administered via the same route (e.g., orally, intravenously, or subcutaneously). In one embodiment, a compound of Formula I (e.g., Compound I), or a pharmaceutically acceptable salt thereof, and a NtRTI or a NRTI (e.g., a compound of Formula II, tenofovir, or Compound II), are each administered orally. In one embodiment, a compound of Formula I (e.g., Compound I), or a pharmaceutically acceptable salt thereof, and a NtRTI or a NRTI (e.g., a compound of Formula II, tenofovir, or Compound II), are administered via different routes. In one embodiment, a NtRTI or a NRTI (e.g., a compound of Formula II, tenofovir, or Compound II), is administered orally, and a compound of Formula I (e.g., Compound I), or a pharmaceutically acceptable salt thereof is administered intravenously.

In one embodiment, a compound of Formula I (e.g., Compound I), or a pharmaceutically acceptable salt thereof, is administered in a therapeutically effective amount. For example Compound I is administered in a therapeutically effective amount. In another embodiment, a compound of Formula I (e.g., Compound I), or a pharmaceutically acceptable salt thereof, is for administration in a therapeutically effective amount. For example, Compound I is for administration in a therapeutically effective amount. In one embodiment, a NtRTI or a NRTI (e.g., a compound of Formula II, tenofovir, or Compound II) is administered in a therapeutically effective amount. In another embodiment, a NtRTI or a NRTI (e.g., a compound of Formula II, tenofovir, or Compound II) is for administration in a therapeutically effective amount. In a further embodiment, a compound of Formula I (e.g., Compound I), or a pharmaceutically acceptable salt thereof, and a NtRTI or a NRTI (e.g., a compound of Formula II, tenofovir, or Compound II), are each administered in a therapeutically effective amount. For example, Compound I and Compound II are each administered in a therapeutically effective amount. In a further embodiment, a compound of Formula I (e.g., Compound I), or a pharmaceutically acceptable salt thereof, and a NtRTI or a NRTI (e.g., a compound of Formula II, tenofovir, or Compound II), are for administration in a therapeutically effective amount. For example, Compound I and Compound II are for administration in a therapeutically effective amount.

In one embodiment, the therapeutically effective amount of a compound of Formula I (e.g., Compound I), or a pharmaceutically acceptable salt thereof, when used in combination with a NtRTI or a NRTI (e.g., a compound of Formula II, tenofovir, or Compound II), is lower in comparison to monotherapy with a compound of Formula I (e.g., Compound I), or a pharmaceutically acceptable salt thereof, without a NtRTI or a NRTI (e.g., a compound of Formula II, tenofovir, or Compound II). For example the therapeutically effective amount of Compound I, or a pharmaceutically acceptable salt thereof, when used in combination with a NtRTI or a NRTI (e.g., a compound of Formula II, tenofovir, or Compound II), is lower in comparison to monotherapy with Compound I, or a pharmaceutically acceptable salt thereof, without a NtRTI or a NRTI (e.g., a compound of Formula II, tenofovir, or Compound II). For example the therapeutically effective amount of Compound I, or a pharmaceutically acceptable salt thereof, when used in combination with a NtRTI or a NRTI (e.g., a compound of Formula II, tenofovir, or Compound II), is reduced by 10%, 20%, 30%, 40%, 50%, 60%, 70%, 80% or 90% in comparison to monotherapy with Compound I, or a pharmaceutically acceptable salt thereof, without a NtRTI or a NRTI (e.g., a compound of Formula II, tenofovir, or Compound II). For example the therapeutically effective amount of Compound I, or a pharmaceutically acceptable salt thereof, when used in combination with a NtRTI or a NRTI (e.g., a compound of Formula II, tenofovir, or Compound II), is reduced by 100%, 200%, 300%, 400%, 500%, 600%, 700%, 800% or 900% in comparison to monotherapy with Compound I, or a pharmaceutically acceptable salt thereof, without a NtRTI or a NRTI (e.g., a compound of Formula II, tenofovir, or Compound II).

In one embodiment, the therapeutically effective amount of a NtRTI or a NRTI (e.g., a compound of Formula II, tenofovir, or Compound II), when used in combination with a compound of Formula I (e.g., Compound I), or a pharmaceutically acceptable salt thereof, is lower in comparison to monotherapy with a NtRTI or a NRTI (e.g., a compound of Formula II, tenofovir, or Compound II), without a compound of Formula I (e.g., Compound I), or a pharmaceutically acceptable salt thereof. For example the therapeutically effective amount of a NtRTI or a NRTI (e.g., a compound of Formula II, tenofovir, or Compound II), when used in combination with Compound I, or a pharmaceutically acceptable salt thereof, is lower in comparison to monotherapy with a NtRTI or a NRTI (e.g., a compound of Formula II, tenofovir, or Compound II), without Compound I, or a pharmaceutically acceptable salt thereof. For example the therapeutically effective amount of a NtRTI or a NRTI (e.g., a compound of Formula II, tenofovir, or Compound II), when used in combination with Compound I, or a pharmaceutically acceptable salt thereof, is reduced by 10%, 20%, 30%, 40%, 50%, 60%, 70%, 80% or 90% in comparison to monotherapy with a NtRTI or a NRTI (e.g., a compound of Formula II, tenofovir, or Compound II), without Compound I, or a pharmaceutically acceptable salt thereof. For example, the therapeutically effective amount of a NtRTI or a NRTI (e.g., a compound of Formula II, tenofovir, or Compound II), when used in combination with Compound I, or a pharmaceutically acceptable salt thereof, is reduced by 100%, 200%, 300%, 400%, 500%, 600%, 700%, 800% or 900% in comparison to monotherapy with a NtRTI or a NRTI (e.g., a compound of Formula II, tenofovir, or Compound II), without Compound I, or a pharmaceutically acceptable salt thereof.

Such lower therapeutically effective amounts offer desirable characteristics, including but not limited to reduce toxicity, of the therapeutic regimen.

In one embodiment, a compound of Formula I (e.g., Compound I), or a pharmaceutically acceptable salt thereof, is administered to a subject, at a dosage from about 100 mg to about 1,000 mg. In one embodiment, a compound of Formula I (e.g., Compound I), or a pharmaceutically acceptable salt thereof, is administered to a subject in an amount of about 100, 150, 200, 250, 300, 350, 400, 450, 500, 600, 700, 800, 900, or 1,000 mg. In one embodiment, a compound of Formula I (e.g., Compound I), or a pharmaceutically acceptable salt thereof, is administered to a subject in an amount of about 100, 150, 200, 250, 300, 350, or 400 mg. In one embodiment, a compound of Formula I (e.g., Compound I), or a pharmaceutically acceptable salt thereof, is administered to the subject in an amount from about 5 mg to about 100 mg. In one embodiment, a compound of Formula I (e.g., Compound I), or a pharmaceutically acceptable salt thereof, is administered to the subject in an amount of about 5, 10, 15, 20, 25, 30, 35, 40, 45, 50, 55, 60, 65, 70, 75, 80, 85, 90, 95, or 100 mg. In some embodiments, a compound of Formula I (e.g., Compound I) is administered at a dose of about 0.5 mg/kg/day, 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, or 20 mg/kg/day.

In one embodiment, a compound of Formula I (e.g., Compound I), or a pharmaceutically acceptable salt thereof, is administered orally. In one embodiment, a compound of Formula I (e.g., Compound I), or a pharmaceutically acceptable salt thereof is administered as a pill, capsule, or tablet. In one embodiment, a compound of Formula I (e.g., Compound I), or a pharmaceutically acceptable salt thereof is administered as a suspension, solution, emulsion, mouthwash, or elixir. In a further embodiment, the subject is a human being.

In one embodiment, a NtRTI or a NRTI (e.g., a compound of Formula II, tenofovir, or Compound II) is administered to a subject, at a dosage from about 100 mg to about 400 mg. In one embodiment, a NtRTI or a NRTI (e.g., a compound of Formula II, tenofovir, or Compound II) is administered to a subject in an amount of about 100, 150, 200, 250, 300, 350, or 400 mg. In one embodiment, a NtRTI or a NRTI (e.g., a compound of Formula II, tenofovir, or Compound II) is administered to the subject in an amount from about 5 mg to about 500 mg. In one embodiment, a NtRTI or a NRTI (e.g., a compound of Formula II, tenofovir, or Compound II) is administered to the subject in an amount of 5, 10, 15, 20, 25, 30, 35, 40, 45, 50, 55, 60, 65, 70, 75, 80, 85, 90, 95, or 100 mg. In one embodiment, a NtRTI or a NRTI (e.g., a compound of Formula II, tenofovir, or Compound II) is administered to the subject in an amount of 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 mg. In some embodiments, a compound of Formula II (e.g., compound II) is administered at a dose of about 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, or 20 mg/kg/day. In one embodiment, a NtRTI or a NRTI (e.g., a compound of Formula II, tenofovir, or Compound II) is administered orally. In one embodiment, a NtRTI or a NRTI (e.g., a compound of Formula II, tenofovir, or Compound II) is administered as a pill, capsule, or tablet. In one embodiment, a NtRTI or a NRTI (e.g., a compound of Formula II, tenofovir, or Compound II) is administered as a suspension, solution, emulsion, mouthwash, or elixir. In a further embodiment, the subject is a human being. In some embodiments, the compound of Formula II (e.g., Compound II) is administered daily, weekly, or every 2 weeks.

Any of the dosages described herein for a compound of Formula I (e.g., Compound I), or a pharmaceutically acceptable salt thereof, can be combined with any of the dosages described herein for a NtRTI or a NRTI (e.g., a compound of Formula II, tenofovir, or Compound II).

(La1) In one embodiment, a compound of Formula I (e.g., Compound I), or a pharmaceutically acceptable salt thereof, is administered for at least a week (e.g., one week, two weeks, three weeks, four weeks, five weeks, six weeks, or more).

(La2) In one embodiment, a compound of Formula I (e.g., Compound I), or a pharmaceutically acceptable salt thereof, is administered for at least a month (e.g., one month, two months, three months, four months, five months, six months, eight months, ten months, twelve months, or more).

(La3) In one embodiment, a compound of Formula I (e.g., Compound I), or a pharmaceutically acceptable salt thereof, is administered for at least three months (e.g., three months, four months, five months, six months, eight months, ten months, twelve months, or more).

(La4) In one embodiment, a compound of Formula I (e.g., Compound I), or a pharmaceutically acceptable salt thereof, is administered for at least six months (e.g., six months, eight months, ten months, twelve months, eighteen months, or more).

(La5) In one embodiment, a compound of Formula I (e.g., Compound I), or a pharmaceutically acceptable salt thereof, is administered for at least a year (e.g., one year, two years, three years, five years, or more).

(La6) In one embodiment, a compound of Formula I (e.g., Compound I), or a pharmaceutically acceptable salt thereof, is administered for at least five years (e.g., five years, six years, seven years, eight years, nine years, ten years, or more).

(La7) In one embodiment, a compound of Formula I (e.g., Compound I), or a pharmaceutically acceptable salt thereof, is administered for at least ten years (e.g., ten years, twelve years, fifteen years, twenty years, or more).

(La8) In one embodiment, a compound of Formula I (e.g., Compound I), or a pharmaceutically acceptable salt thereof, is administered for the duration of the subject's life.

(Lb1) In one embodiment, a NtRTI or a NRTI (e.g., a compound of Formula II, tenofovir, or Compound II) is administered for at least a week (e.g., one week, two weeks, three weeks, four weeks, five weeks, six weeks, or more).

(Lb2) In one embodiment, a NtRTI or a NRTI (e.g., a compound of Formula II, tenofovir, or Compound II) is administered for at least a month (e.g., one month, two months, three months, four months, five months, six months, eight months, ten months, twelve months, or more).

(Lb3) In one embodiment, a NtRTI or a NRTI (e.g., a compound of Formula II, tenofovir, or Compound II) is administered for at least three months (e.g., three months, four months, five months, six months, eight months, ten months, twelve months, or more).

(Lb4) In one embodiment, a NtRTI or a NRTI (e.g., a compound of Formula II, tenofovir, or Compound II) is administered for at least six months (e.g., six months, eight months, ten months, twelve months, eighteen months, or more).

(Lb5) In one embodiment, a NtRTI or a NRTI (e.g., a compound of Formula II, tenofovir, or Compound II) is administered for at least a year (e.g., one year, two years, three years, five years, or more).

(Lb6) In one embodiment, a NtRTI or a NRTI (e.g., a compound of Formula II, tenofovir, or Compound II) is administered for at least five years (e.g., five years, six years, seven years, eight years, nine years, ten years, or more).

(Lb7) In one embodiment, a NtRTI or a NRTI (e.g., a compound of Formula II, tenofovir, or Compound II) is administered for at least ten years (e.g., ten years, twelve years, fifteen years, twenty years, or more).

(Lb8) In one embodiment, a NtRTI or a NRTI (e.g., a compound of Formula II, tenofovir, or Compound II) is administered for the duration of the subject's life.

Any of the treatment duration described herein for a compound of Formula I (e.g., Compound I), or a pharmaceutically acceptable salt thereof (e.g., (La1)-(La8)), can be combined with any of the treatment duration described herein for a NtRTI or a NRTI (e.g., a compound of Formula II, tenofovir, or Compound II) (e.g., (Lb1)-(Lb8)).

In one embodiment, a compound of Formula I (e.g., Compound I), or a pharmaceutically acceptable salt thereof, and a NtRTI or a NRTI (e.g., a compound of Formula II, tenofovir, or Compound II), are administered with the same treatment duration as described in (La1)-(La8) and (Lb1)-(Lb8), respectively (e.g., a compound of Formula I (e.g., Compound I), or a pharmaceutically acceptable salt thereof, is administered according to (La1), and a NtRTI or a NRTI (e.g., a compound of Formula II, tenofovir, or Compound II), is administered according to (Lb1)).

(Ra1) In one embodiment, a compound of Formula I (e.g., Compound I), or a pharmaceutically acceptable salt thereof, is administered at least three times (e.g., three times, four times, or more) daily.

(Ra2) In one embodiment, a compound of Formula I (e.g., Compound I), or a pharmaceutically acceptable salt thereof, is administered twice daily.

(Ra3) In one embodiment, a compound of Formula I (e.g., Compound I), or a pharmaceutically acceptable salt thereof, is administered once daily.

(Ra4) In one embodiment, a compound of Formula I (e.g., Compound I), or a pharmaceutically acceptable salt thereof, is administered once every two days.

(Ra5) In one embodiment, a compound of Formula I (e.g., Compound I), or a pharmaceutically acceptable salt thereof, is administered once every three days.

(Ra6) In one embodiment, a compound of Formula I (e.g., Compound I), or a pharmaceutically acceptable salt thereof, is administered once weekly.

(Ra7) In one embodiment, a compound of Formula I (e.g., Compound I), or a pharmaceutically acceptable salt thereof, is administered for 1, 2, 3, 4, 5, 6, or 7 days per week.

(Ra8) In one embodiment, a compound of Formula I (e.g., Compound I), or a pharmaceutically acceptable salt thereof, is administered for 1, 2, 3, 4, 5, or 6 days per week.

(Ra9) In one embodiment, a compound of Formula I (e.g., Compound I), or a pharmaceutically acceptable salt thereof, is administered for 2, 3, 4, 5, 6, or 7 days per week.

(Ra10) In one embodiment, a compound of Formula I (e.g., Compound I), or a pharmaceutically acceptable salt thereof, is administered for 2, 3, 4, 5, or 6 days per week.

(Ra11) In one embodiment, a compound of Formula I (e.g., Compound I), or a pharmaceutically acceptable salt thereof, is administered once, twice, or three times daily continuously for more than one day per week, followed by discontinuation of the administration for the rest of the week.

(Ra12) In one embodiment, a compound of Formula I (e.g., Compound I), or a pharmaceutically acceptable salt thereof, is administered once, twice, or three times daily every other day.

(Ra13) In one embodiment, a compound of Formula I (e.g., Compound I), or a pharmaceutically acceptable salt thereof, is administered once, twice, or three times daily every three days, every four days, every five days, every six days, or every seven days.

(Ra14) In one embodiment, a compound of Formula I (e.g., Compound I), or a pharmaceutically acceptable salt thereof, is administered once, twice, or three times daily for two days in a row every three days, every four days, every five days, every six days, or every seven days.

(Ra15) In one embodiment, a compound of Formula I (e.g., Compound I), or a pharmaceutically acceptable salt thereof, is administered once, twice, or three times daily for three days in a row every four days, every five days, every six days, or every seven days.

(Ra16) In one embodiment, a compound of Formula I (e.g., Compound I), or a pharmaceutically acceptable salt thereof, is administered once, twice, or three times daily for four days in a row every five days, every six days, or every seven days.

(Rb1) In one embodiment, a NtRTI or a NRTI (e.g., a compound of Formula II, tenofovir, or Compound II) is administered at least three times (e.g., three times, four times, or more) daily.

(Rb2) In one embodiment, a NtRTI or a NRTI (e.g., a compound of Formula II, tenofovir, or Compound II) is administered twice daily.

(Rb3) In one embodiment, a NtRTI or a NRTI (e.g., a compound of Formula II, tenofovir, or Compound II) is administered once daily.

(Rb4) In one embodiment, a NtRTI or a NRTI (e.g., a compound of Formula II, tenofovir, or Compound II) is administered once every two days.

(Rb5) In one embodiment, a NtRTI or a NRTI (e.g., a compound of Formula II, tenofovir, or Compound II) is administered once every three days.

(Rb6) In one embodiment, a NtRTI or a NRTI (e.g., a compound of Formula II, tenofovir, or Compound II) is administered once weekly.

(Rb7) In one embodiment, a NtRTI or a NRTI (e.g., a compound of Formula II, tenofovir, or Compound II) is administered for 1, 2, 3, 4, 5, 6, or 7 days per week.

(Rb8) In one embodiment, a NtRTI or a NRTI (e.g., a compound of Formula II, tenofovir, or Compound II) is administered for 1, 2, 3, 4, 5, or 6 days per week.

(Rb9) In one embodiment, a NtRTI or a NRTI (e.g., a compound of Formula II, tenofovir, or Compound II) is administered for 2, 3, 4, 5, 6, or 7 days per week.

(Rb10) In one embodiment, a NtRTI or a NRTI (e.g., a compound of Formula II, tenofovir, or Compound II) is administered for 2, 3, 4, 5, or 6 days per week.

(Rb11) In one embodiment, a NtRTI or a NRTI (e.g., a compound of Formula II, tenofovir, or Compound II) is administered once, twice, or three times daily continuously for more than one day per week, followed by discontinuation of the administration for the rest of the week.

(Rb12) In one embodiment, a NtRTI or a NRTI (e.g., a compound of Formula II, tenofovir, or Compound II) is administered once, twice, or three times daily every other day.

(Rb13) In one embodiment, a NtRTI or a NRTI (e.g., a compound of Formula II, tenofovir, or Compound II) is administered once, twice, or three times daily every three days, every four days, every five days, every six days, or every seven days.

(Rb14) In one embodiment, a NtRTI or a NRTI (e.g., a compound of Formula II, tenofovir, or Compound II) is administered once, twice, or three times daily for two days in a row every three days, every four days, every five days, every six days, or every seven days.

(Rb15) In one embodiment, a NtRTI or a NRTI (e.g., a compound of Formula II, tenofovir, or Compound II) is administered once, twice, or three times daily for three days in a row every four days, every five days, every six days, or every seven days.

(Rb16) In one embodiment, a NtRTI or a NRTI (e.g., a compound of Formula II, tenofovir, or Compound II) is administered once, twice, or three times daily for four days in a row every five days, every six days, or every seven days.

Any of the dosing frequencies described herein for a compound of Formula I (e.g., Compound I), or a pharmaceutically acceptable salt thereof (e.g., (Ra1)-(Ra16)), can be combined with any of the dosing frequencies described herein for a NtRTI or a NRTI (e.g., a compound of Formula II, tenofovir, or Compound II) (e.g., (Rb1)-(Rb16)).

In one embodiment, a compound of Formula I (e.g., Compound I), or a pharmaceutically acceptable salt thereof, and a NtRTI or a NRTI (e.g., a compound of Formula II, tenofovir, or Compound II), are administered with the same dosing frequency as described in (Ra1)-(Ra16) and (Rb1)-(Rb16), respectively (e.g., a compound of Formula I (e.g., Compound I),

or a pharmaceutically acceptable salt thereof, is administered according to (Ra1), and a NtRTI or a NRTI (e.g., a compound of Formula II, tenofovir, or Compound II), is administered according to (Rb1)).

In one embodiment, for any of the compounds described herein (e.g., Compound I or Compound II), the treatment duration is independent of the dosing frequency (i.e., the dosing frequency may change from time to time while the treatment is still ongoing).

In one embodiment, for any of the compounds described herein (e.g., Compound I or Compound II), the treatment duration is independent of the dosage (i.e., the dosage may change from time to time while the treatment is still ongoing).

In one embodiment, for any of the compounds described herein (e.g., Compound I or Compound II), the dosing frequency is independent of the dosage (i.e., the dosage may change from time to time while the dosing frequency remains the same, and vice versa).

A HBV disease is a disease or condition caused by or associated with HBV infection. A HBV disease includes chronic HBV infection, hepatitis B, liver fibrosis, cirrhosis, and hepatocellular carcinoma. In one embodiment, a HBV disease is hepatitis B. In one embodiment, a HBV disease is chronic HBV infection. As used herein “HBV” is intended to include all subtypes (adw, adr, ayw, and ayr) and/or genotypes (A, B, C, D, E, F, G, and H) thereof.

“Human immunodeficiency virus” (or “HIV”), as used herein, is intended to include all groups (e.g., groups M, N, O, and P) and subtypes (e.g., HIV subtypes A, B, C, D, E, F, G, H, J, K, and 0) thereof.

A HIV disease is a disease or condition caused by or associated with HIV infection. A HIV disease includes acquired immunodeficiency syndrome (AIDS) and conditions and disorders related to AIDS, such as AIDS related cancers (e.g., Kaposi's sarcoma, lymphoma), AIDS related infections (e.g., tuberculosis, candidiasis, cryptococcal meningitis, toxoplasmosis, and cryptosporidiosis), wasting syndrome, neurological complications, and kidney disease.

As used herein, a “nucleoside analog reverse transcriptase inhibitor” may also be called “nucleoside reverse transcriptase inhibitor”, or “NRTI”, or “NARTI”.

As used herein, a “nucleotide analog reverse transcriptase inhibitor” may also be called “nucleotide reverse transcriptase inhibitor”, or “NtRTI”, or “NtARTI”.

“Carboxylic acid” has the formula COOH, but may include a group in which the carboxyl moiety is connected to one of the following groups:

alkyl which may be substituted (for example, alkyl of 2 to 15 carbons);

alkenyl which may be substituted (for example, alkenyl of 2 to 15 carbons); and

alkynyl which may be substituted (for example, alkynyl of 2 to 15 carbons).

The substituents as described herein may include halogen (for example, fluorine, chlorine, bromine, iodine, etc.), nitro, cyano, hydroxy, thiol which may be substituted (for example, thiol, C₁-C₄ alkylthio, etc.), amino which may be substituted (for example, amino, mono-C₁-C₄ alkylamino, di-C₁-C₄ alkylamino, 5- to 6-membered cyclic amino such as tetrahydropyrrole, piperazine, piperidine, morpholine, thiomorpholine, pyrrole, imidazole, etc.), C₁-C₄ alkoxy which may be halogenated (for example, methoxy, ethoxy, propoxy, butoxy, trifluoromethoxy, trifluoroethoxy, etc.), C₁-C₄ alkoxy-C₁-C₄ alkoxy which may be halogenated (for example, methoxymethoxy, methoxyethoxy, ethoxyethoxy, trifluoromethoxyethoxy, trifluoroethoxyethoxy, etc.), formyl, C₂-C₄ alkanoyl (for example, acetyl, propionyl, etc.), C₁-C₄ alkylsulfonyl (for example, methanesulfonyl, ethanesulfonyl, etc.), and the like, and the number of the substituents is preferably 1 to 3.

Further, the substituents of the above “amino which may be substituted” may bind each other to form a cyclic amino group (for example, a group which is formed by subtracting a hydrogen atom from the ring constituting nitrogen atom of a 5- to 6-membered ring such as tetrahydropyrrole, piperazine, piperidine, morpholine, thiomorpholine, pyrrole, imidazole, etc. so that a substituent can be attached to the nitrogen atom, or the like). The cyclic amino group may be substituted and examples of the sub stituent include halogen (for example, fluorine, chlorine, bromine, iodine, etc.), nitro, cyano, hydroxy, thiol which may be substituted (for example, thiol, C₁-C₄ alkylthio, etc.), amino which may be substituted (for example, amino, mono-C₁-C₄ alkylamino, di-C₁-C₄ alkylamino, 5- to 6-membered cyclic amino such as tetrahydropyrrole, piperazine, piperidine, morpholine, thiomorpholine, pyrrole, imidazole, etc.), carboxyl which may be esterified or amidated (for example, carboxyl, C₁-C₄ alkoxy-carbonyl, carbamoyl, mono-C₁-C₄ alkyl-carbamoyl, di-C₁-C₄ alkyl-carbamoyl, etc.), C₁-C₄ alkoxy which may be halogenated (for example, methoxy, ethoxy, propoxy, butoxy, trifluoromethoxy, trifluoroethoxy, etc.), C₁-C₄ alkoxy-C₁-C₄ alkoxy which may halogenated (for example, methoxymethoxy, methoxyethoxy, ethoxyethoxy, trifluoromethoxyethoxy, trifluoroethoxyethoxy, etc.), formyl, C₂₋₄ alkanoyl (for example, acetyl, propionyl, etc.), C₁-C₄ alkylsulfonyl (for example, methanesulfonyl, ethanesulfonyl), and the like, and the number of the substituents is preferably 1 to 3.

“Amine” includes a group which may be unsubstituted or in which the amine moiety is N-substituted or N,N disubstituted having one or two substituents which may be independently selected from:

alkyl which may be substituted (for example, alkyl of 2 to 15 carbons);

alkenyl which may be substituted (for example, alkenyl of 2 to 15 carbons);

alkynyl which may be substituted (for example, alkynyl of 2 to 15 carbons);

formyl or acyl which may be substituted (for example, alkanoyl of 2 to 4 carbons (for example, acetyl, propionyl, butyryl, isobutyryl, etc.), alkylsulfonyl of 1 to 4 carbons (for example, methanesulfonyl, ethanesulfonyl, etc.), and the like);

aryl which may be substituted (for example, phenyl, naphthyl, etc.); and the like;

and connected to a substituent independently selected from the substituents as defined above (e.g., for “carboxylic acid”).

“Amide” includes a linkage of the formula —C(O)NR₂—. For example, amides include compounds in which the carbonyl group of the amide moiety is connected to a substituent independently selected from the substituents as defined above (e.g., for “carboxylic acid”). In some embodiments, the amino group of the amide moiety is an N-substituted or N,N disubstituted amine having one or two substituents, respectively, which may be independently selected from:

alkyl which may be substituted (for example, alkyl of 2 to 15 carbons);

alkenyl which may be substituted (for example, alkenyl of 2 to 15 carbons);

alkynyl which may be substituted (for example, alkynyl of 2 to 15 carbons);

formyl or acyl which may be substituted (for example, alkanoyl of 2 to 4 carbons (for example, acetyl, propionyl, butyryl, isobutyryl, etc.), alkylsulfonyl of 1 to 4 carbons (for example, methanesulfonyl, ethanesulfonyl, etc.) and the like);

aryl which may be substituted (for example, phenyl, naphthyl, etc.); and the like.

“Aryl” may be exemplified by a monocyclic or fused polycyclic aromatic hydrocarbon group, and for example, a C₆-C₁₄ aryl group such as phenyl, naphthyl, anthryl, phenanthryl or acenaphthylenyl, and the like are preferred, with phenyl being preferred. Said aryl may be substituted with one or more substituents, such as lower alkoxy (e.g., C₁-C₆ alkoxy such as methoxy, ethoxy or propoxy, etc.), a halogen atom (e.g., fluorine, chlorine, bromine, iodine, etc.), lower alkyl (e.g., C₁-C₆ alkyl such as methyl, ethyl or propyl, etc.), lower alkenyl (e.g., C₂-C₆ alkenyl such as vinyl or allyl, etc.), lower alkynyl (e.g., C₂-C₆ alkynyl such as ethynyl or propargyl, etc.), amino which may be substituted, hydroxyl which may be substituted, cyano, amidino which may be substituted, carboxyl, lower alkoxycarbonyl (e.g., C₁-C₆ alkoxycarbonyl such as methoxycarbonyl or ethoxycarbonyl, etc.), carbamoyl which may be substituted (e.g., carbamoyl which may be substituted with C₁-C₆ alkyl or acyl (e.g., formyl, C₂-C₆ alkanoyl, benzoyl, C₁-C₆ alkoxycarbonyl which may be halogenated, C₁-C₆ alkylsulfonyl which may be halogenated, benzenesulfonyl, etc.) which may be substituted with a 5- to 6-membered aromatic monocyclic heterocyclic group (e.g., pyridinyl, etc.), 1-azetidinylcarbonyl, 1-pyrrolidinylcarbonyl, piperidinocarbonyl, morpholinocarbonyl, thiomorpholinocarbonyl (the sulfur atom may be oxidized), 1-piperazinylcarbonyl, etc.), and the like. Any of these substituents may be independently substituted at 1 to 3 substitutable positions.

“Ketone” includes a compound in which the carbonyl group of the ketone moiety is connected to one or two substituents independently selected from the substituents as defined above (e.g., for “carboxylic acid”).

The term “carbonyl” refers to a functional group composing a carbon atom double-bonded to an oxygen atom. It can be abbreviated herein as “oxo”, as C(O), or as C═O.

“Ester” refers to a linkage of the formula —RC(O)OR′—. The term “ester” includes either a carboxylic or an alcohol ester wherein of the ester group is composed of one or two substituents independently selected from the substituents as defined above (e.g., for “carboxylic acid” or for “aryl”).

A “nitrile” refers to a group of the formula —CN.

A “hydroxy” refers to a group of the formula —OH.

“Dioxolane’ is a heterocyclic acetal with the chemical formula (CH₂)ₙO₂CH₂, wherein n may be, e.g., 2 or 3.

“Alkyl” unless otherwise defined is an alkyl of 1 to 15 carbon units in length. In one embodiment, “alkyl” is an alkyl of 1 to 6 carbon units, 1 to 5 carbon units, 1 to 4 carbon units, or 1 to 3 carbon units (e.g., methyl, ethyl, propyl, i-propyl, butyl, i-butyl, s-butyl, t-butyl, pentyl, or hexyl).

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propanyl, n-butynyl, iso-butynyl, pentynyl, or hexynyl. An alkynyl group can be unsubstituted or substituted.

An “aliphatic chain” refers to a hydrocarbon chain that may be saturated or unsaturated. Aliphatic chains may be substituted as set forth herein. Exemplary aliphatic chains include alkyl groups, alkenyl groups, and alkynyl groups.

“Aromatic group” may be exemplified by aryl as defined above, or a 5- to 6-membered aromatic monocyclic heterocyclic group such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl or the like; and a 8- to 16-membered (e.g., 10- to 12-membered) aromatic fused heterocyclic group.

“Non-immunosuppressive” refers to the ability of a compound to exhibit a substantially reduced level of suppression of the immune system as compared with CsA, as measured by the compound's ability to inhibit the proliferation of human lymphocytes in cell culture, for example, as measured by the method set out in the Examples.

“Analogue” or “analog” means a structural analogue of CsA that differs from CsA in one or more functional groups. For example, such analogues preserve at least a substantial portion of the ability of CsA to bind CyP.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

“Treat”, “treating” and “treatment” refer to a method of alleviating or abating a disease and/or its attendant symptoms.

As used herein, “preventing” or “prevent” describes reducing or eliminating the onset of the symptoms or complications of the disease, condition or disorder.

The terms “disease(s)”, “disorder(s)”, and “condition(s)” are used interchangeably, unless the context clearly dictates otherwise.

The term “therapeutically effective amount” of a compound or pharmaceutical composition of the application, as used herein, means a sufficient amount of the compound or pharmaceutical composition so as to decrease the symptoms of a disorder in a subject. The term “therapeutically effective amount” of a compound or pharmaceutical composition of the application, as used herein, may mean a sufficient amount of the compound or pharmaceutical composition so as to slow or halt progression of a HBV disease (e.g., hepatitis B, cirrhosis, or hepatocellular carcinoma) or a HIV disease (e.g., AIDS). The term “therapeutically effective amount” of a compound or pharmaceutical composition of the application, as used herein, may mean a sufficient amount of the compound or pharmaceutical composition so as to slow or halt progression of a HBV disease to cirrhosis or hepatocellular carcinoma) or a HIV disease. The term “therapeutically effective amount” of a compound or pharmaceutical composition of the application, as used herein, may also mean a sufficient amount of the compound or pharmaceutical composition so as to improve symptoms of an HBV disease or a HIV disease. The term “therapeutically effective amount” of a compound or pharmaceutical composition of the application, as used herein, may mean a sufficient amount of the compound or pharmaceutical composition so as to reduce HBV or HIV viral load, or modulate (e.g., inhibit or decrease) replication of HBV (e.g., by inhibiting replication of HBV DNA in a cell, such as an HBV infected cell) or HIV (e.g., by inhibiting replication of HIV DNA in a cell, such as an HIV infected cell).

As is well understood in the medical arts a therapeutically effective amount of a compound or pharmaceutical composition of this application will be at a reasonable benefit/risk ratio applicable to any medical treatment. It will be understood, however, that the total daily usage of the compounds and compositions of the present application will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

As used herein, the term “pharmaceutically acceptable salt” refers to those salts of the compounds formed by the process of the present application which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the application, or separately by reacting the free base or acid function with a suitable acid or base.

Examples of pharmaceutically acceptable salts include, but are not limited to, nontoxic acid addition salts: salts formed with inorganic acids such as hydrochloric acid, hydrobromic acid, phosphoric acid, sulfuric acid and perchloric acid, or with organic acids such as acetic acid, maleic acid, tartaric acid, citric acid, succinic acid or malonic acid. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

The compounds of the present application may exist in the form of optically active compounds. The present application contemplates all enantiomers of optically active compounds within the scope of the above formulae, both individually and in mixtures of racemates. As well, the present application includes prodrugs of the compounds defined herein.

Optical isomers may be prepared from their respective optically active precursors by the procedures described herein, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981).

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Epimer” means one member of a pair of stereoisomers wherein the two isomers differ in configuration at only one stereogenic center and all other stereocenters in the molecules, if any, are the same in each.

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine. The compounds of this application may also be represented in multiple tautomeric forms, in such instances, the application expressly includes all tautomeric forms of the compounds described herein (e.g., alkylation of a ring system may result in alkylation at multiple sites, the application expressly includes all such reaction products).

In the present application, the structural formula of the compound represents a certain isomer for convenience in some cases, but the present application includes all isomers, such as geometrical isomers, optical isomers based on an asymmetrical carbon, stereoisomers, tautomers, and the like. In the present specification, the structural formula of the compound represents a certain isomer for convenience in some cases, but the present application includes all isomers, such as geometrical isomers, optical isomers based on an asymmetrical carbon, stereoisomers, tautomers, and the like.

In some embodiments, “temporal proximity” means that administration of one therapeutic agent occurs within a time period before or after the administration of another therapeutic agent, such that the therapeutic effect of the one therapeutic agent overlaps with the therapeutic effect of the another therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the another therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

According to one aspect, a compound of this application may be administered neat or with a pharmaceutical carrier to a warm-blooded animal in need thereof. The pharmaceutical carrier may be solid or liquid. The compound may be administered orally, topically, parenterally, by inhalation spray or rectally in dosage unit formulations containing conventional non-toxic pharmaceutically acceptable carriers, adjuvants and vehicles. The term parenteral, as used herein, includes subcutaneous injections, intravenous, intramuscular, intrasternal injection or infusion techniques.

The pharmaceutical compositions containing the inventive mixture may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions and such compositions may contain one or more agents selected from the group consisting of sweetening agents, flavoring agents, coloring agents and preserving agents in order to provide pharmaceutically elegant and palatable preparation. Tablets containing the active ingredient in admixture with non-toxic pharmaceutically acceptable excipients may also be manufactured by known methods. The excipients used may be for example, (1) inert diluents such as calcium carbonate, lactose, calcium phosphate or sodium phosphate; (2) granulating and disintegrating agents such as corn starch, or alginic acid; (3) binding agents such as starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,160,452; and U.S. Pat. No. 4,265,874 to form osmotic therapeutic tablets for controlled release.

In some cases, formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, for example peanut oil, liquid paraffin, or olive oil.

Aqueous suspensions normally contain the active materials in admixture with excipients suitable for the manufacture of aqueous suspensions. Such excipients may include: (1) suspending agents such as sodium carboxymethylcellulose, methylcellulose, hydroxypropylmethylcellulose, sodium alginate, polyvinylpyrrolidone, gum tragacanth and gum acacia; or (2) dispersing or wetting agents which may be a naturally-occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived from a fatty acid and a hexitol such as polyoxyethylene sorbitol monooleate, or a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents such as sucrose, aspartame or saccharin.

Oily suspension may be formulated by suspending the active ingredient in a vegetable oil, for example arachis oil, olive oil, sesame oil or coconut oil, a fish oil which contains omega 3 fatty acid, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be preserved by the addition of an antioxidant such as ascorbic acid.

Dispersible powders and granules are suitable for the preparation of an aqueous suspension. They provide the active ingredient in a mixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions containing the inventive mixture may also be in the form of oil-in-water emulsions. The oily phase may be a vegetable oil such as olive oil or arachis oils, or a mineral oil such as liquid paraffin or a mixture thereof. Suitable emulsifying agents may be (1) naturally-occurring gums such as gum acacia and gum tragacanth, (2) naturally-occurring phosphatides such as soy bean and lecithin, (3) esters or partial ester 30 derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Syrups and elixirs may be formulated with sweetening agents, for example, glycerol, propylene glycol, sorbitol, aspartame or sucrose. Such formulations may also contain a demulcent, a preservative, and flavoring and coloring agents.

The pharmaceutical compositions may be in the form of a sterile injectable aqueous or oleagenous suspension. This suspension may be formulated according to known methods using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

The inventive compound may also be administered in the form of suppositories for rectal administration of the drug. Suitable compositions can be prepared by mixing the compound with a suitable non-irritating excipient which is solid at ordinary temperatures but liquid at the rectal temperature and will therefore melt in the rectum to release the drug. Such materials are cocoa butter and polyethylene glycols.

For topical use, suitable creams, ointments, jellies, solutions or suspensions, etc., which normally are used with cyclosporine may be employed.

In a particularly preferred embodiment, a liquid solution containing a surfactant, ethanol, a lipophilic and/or an amphiphilic solvent as non-active ingredients is used. Specifically, an oral multiple emulsion formula containing the isomeric analogue mixture and the following non-medicinal ingredients: d-alpha Tocopheryl polyethylene glycol 1000 succinate (vitamin E TPGS), medium chain triglyceride (MCT) oil, Tween 40, and ethanol is used. A soft gelatin capsule (comprising gelatin, glycerin, water, and sorbitol) containing the compound and the same non-medicinal ingredients as the oral solution may also preferably be used.

It will be understood, however, that the specific dose level for any particular patient will depend upon a variety of factors including the activity of the specific compound employed, the age, body weight, general health, sex, diet, time of administration, route of administration, rate of excretion, drug combination and the nature and severity of the particular disease or condition undergoing therapy.

As shown in Examples 3 and 4 below, compounds of Formula I (e.g., Compound I) and compounds of Formula II (e.g., Compound II) can have a synergistic effect on the replication of viruses (e.g., hepatitis B virus). As shown in Example 4, additive or greater reductions in HBV DNA were achieved using a combination of Compound I and Compound II.

### EXAMPLES

### Example 1. Synthesis of Compounds of the Present Application

Compounds of the present application may be prepared according to methods known in the art, for example WO 2012/079172, the contents of which are incorporated herein in their entirety. Exemplary reactions are illustrated below.

### Wittig Reaction

The Wittig reaction is broadly applicable to a wide range of substrates and reactants. The side chain, which is introduced to the substrate in the reaction, can represent any number of branched and unbranched, saturated and unsaturated aliphatic compounds of variable length (R′) and may contain a broad range of functional groups. In the Wittig reaction, a base, such as potassium tert-butoxide (KOtBu) is used to generate an ylide from a phosphonium salt. The ylide reacts with the carbonyl group of the substrate, CsA-aldehyde, to form an alkene. Phosphonium salts containing a carboxylic acid side chain require at least two equivalents of base to generate the ylide.

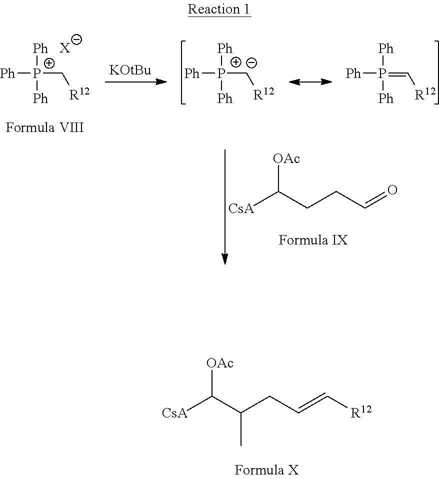

wherein X is a halide (including but not limited to Cl, Br, and I), and R¹² is a saturated or unsaturated straight or branched aliphatic carbon chain, optionally containing a substituent selected from the group of ketones, hydroxyls, nitriles, carboxylic acids, esters, and 1,3-dioxolanes; an aromatic group, optionally containing a substituent selected from the group of halides, esters, and nitro; or a combination of the aforementioned saturated or unsaturated straight or branched aliphatic carbon chain and the aforementioned aromatic groups.

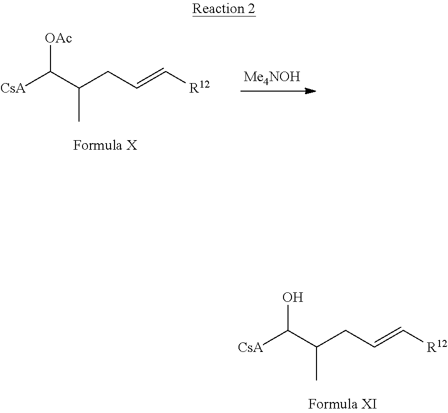

wherein R¹² is a saturated or unsaturated straight or branched aliphatic carbon chain, optionally containing a substituent selected from the group of ketones, hydroxyls, nitriles, carboxylic acids, esters, amides, acyl-protected amines, and 1,3-dioxolanes; an aromatic group, optionally containing a substituent selected from the group of halides, esters, amines, and nitro; or a combination of the aforementioned saturated or unsaturated straight or branched aliphatic carbon chain and the aforementioned aromatic groups.

Using Reaction 2, the following compounds were synthesized.

| Compound | Starting Material | MS (Na⁺) |
|---|---|---|
| 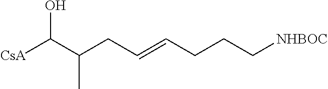 | 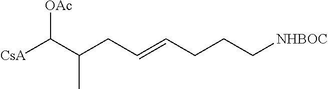 | 1368.0 |
| 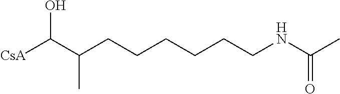 | 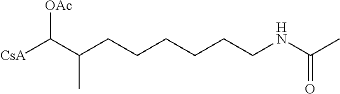 | 1312.0 |
| 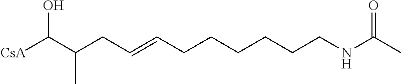 | 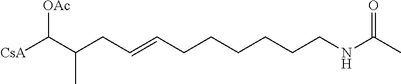 | 1352.1 |
| 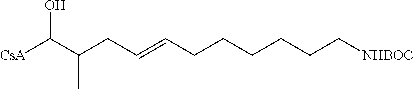 | 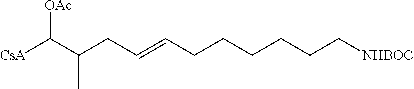 | 1410.0 |
| 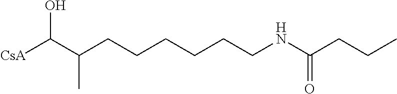 | 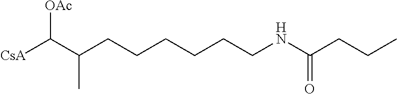 | 1340.0 |
| 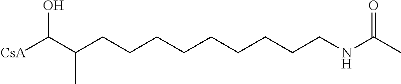 | 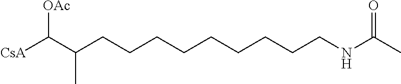 | 1354.0 |
| 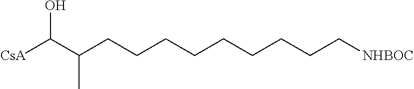 | 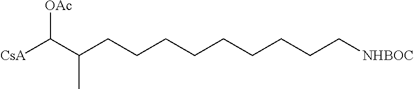 | 1412.0 |
| 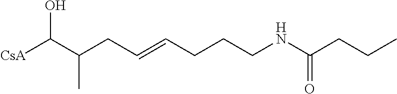 | 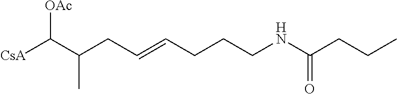 | 1337.9 |
| 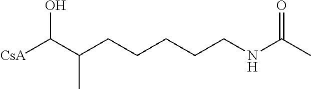 | 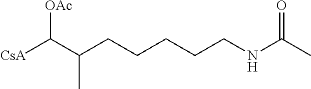 | 1297.9 |
| 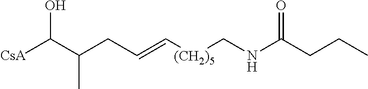 | 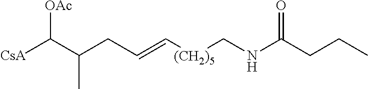 | 1380.0 |
| 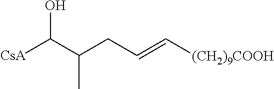 | 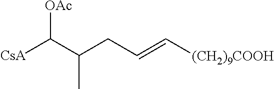 | 1381.1 |
| 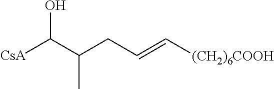 | 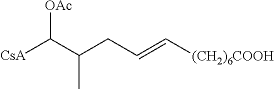 | 1339.1 |
| 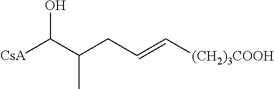 | 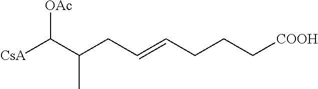 | 1297.1 |
| 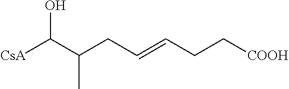 | 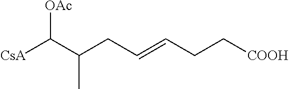 | 1283.0 |
| 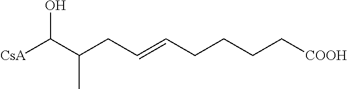 | 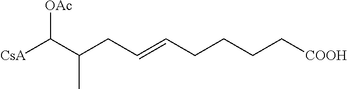 | 1311.0 |

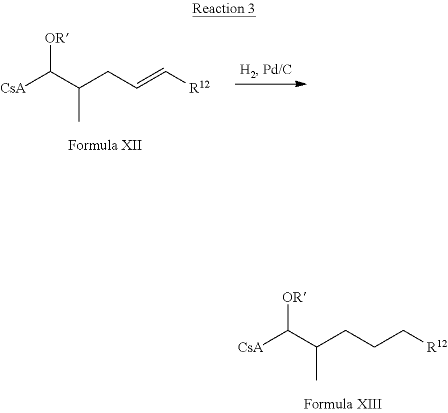

wherein R¹² is a saturated or unsaturated straight or branched aliphatic carbon chain, optionally containing a substituent selected from the group of ketones, hydroxyls, nitriles, carboxylic acids, esters, amides, acyl-protected amines, and 1,3-dioxolanes; an aromatic group, optionally containing a substituent selected from the group of halides, esters, amines and nitro; or a combination of the aforementioned saturated or unsaturated straight or branched aliphatic carbon chain and the aforementioned aromatic groups, and R′ is H or acetyl.

Using Reaction 3, the following compounds were synthesized.

| Compound | Starting Material | MS (Na⁺) |
|---|---|---|
| 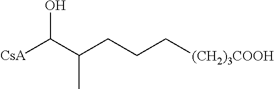 | 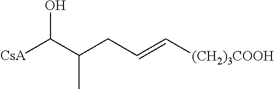 | 1299.1 |
| 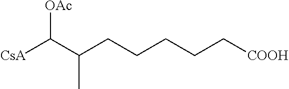 | 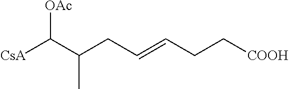 | 1326.8 |
| 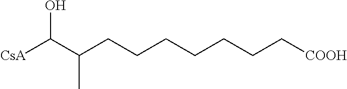 | 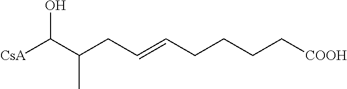 | 1313.0 |
| 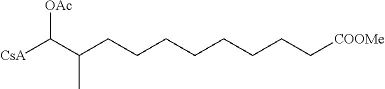 | 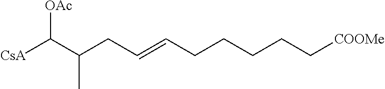 | 1383.1 |
| 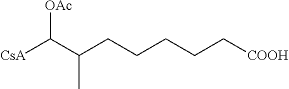 | 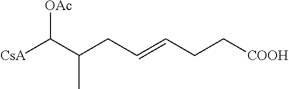 | 1326.9 |
| 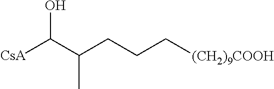 | 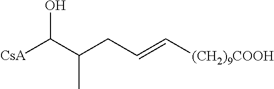 | 1383.1 |
| 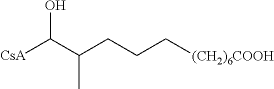 | 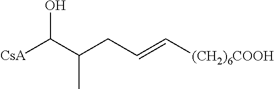 | 1341.0 |
| 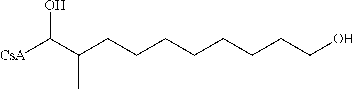 | 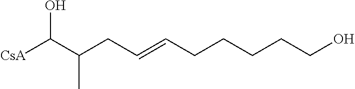 | 1298.9 |
| 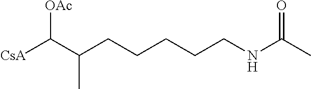 | 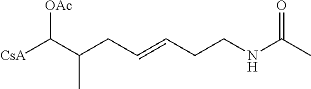 | 1340.0 |

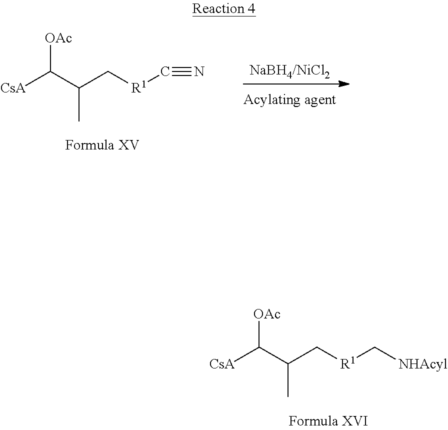

wherein “Acyl” is any one of BOC, acetyl, or butyryl, acylating agent is any one of di-tert-butyldicarbonate, acetic anhydride, and butyric anhydride and R¹ is a saturated or unsaturated straight or branched aliphatic group. It would be understood by one skilled in the art that the acylating agents described above may be replaced with a broad range of acylating agents to produce a similarly broad range of acyl-protected amines.

Using Reaction 4, the following compounds were synthesized.

| | | Protecting | MS |
|---|---|---|---|
| Compound | Starting Material | Reagent | (Na⁺) |
| 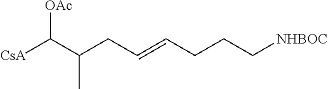 | 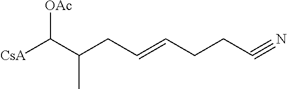 | di-tert- butyldicarbonate | 1410.0 |
| 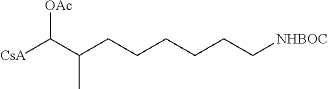 | 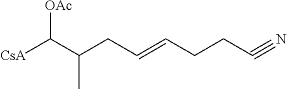 | di-tert- butyldicarbonate | 1412.1 |
| 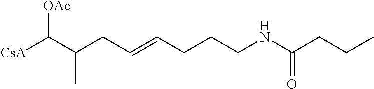 | 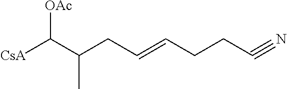 | butyric anhydride | 1379.9 |
| 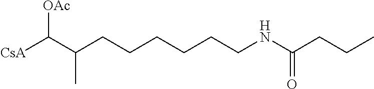 | 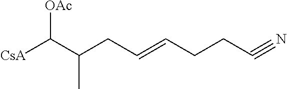 | butyric anhydride | 1382.1 |
| 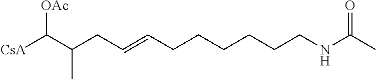 | 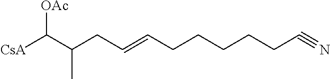 | acetic anhydride | 1394.1 |
| 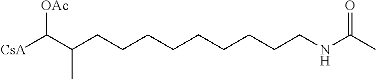 | 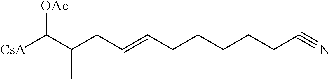 | acetic anhydride | 1396.1 |
| 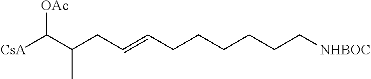 | 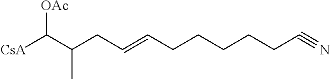 | di-tert- butyldicarbonate | 1452.1 |
| 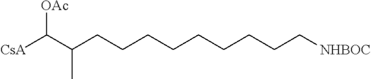 | 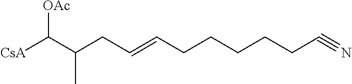 | di-tert- butyldicarbonate | 1454.1 |
| 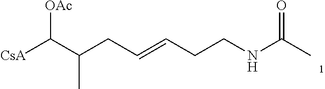 | 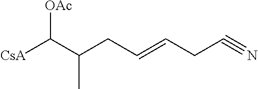 | acetic anhydride | 1337.9/ 1339.9 |
| 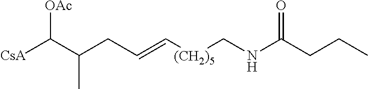 | 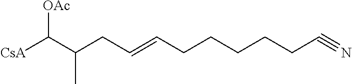 | butyric anhydride | 1422.1 |
| 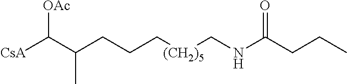 | 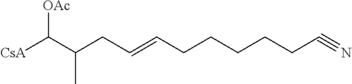 | butyric anhydride | 1424.1 |

¹ mixture not separated

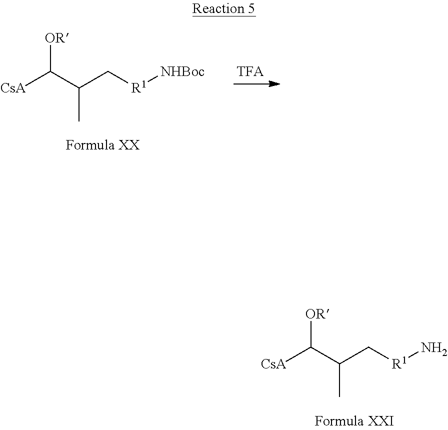

wherein R¹ is a saturated or unsaturated straight or branched aliphatic carbon chain, and R′ is H or acetyl.

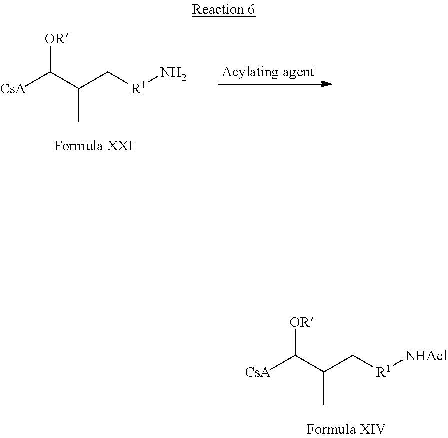

wherein Acyl is any one of BOC, acetyl or butyryl, acylating agent is any one of di-tert-butyldicarbonate, acetic anhydride, or butyric anhydride. It would be understood by one skilled in the art that a broad range of acylating agents including, dicarbonates, anhydrides and acyl halides can be employed to produce a broad range of acyl-protected amines, and R¹ is a saturated or unsaturated straight or branched aliphatic group.

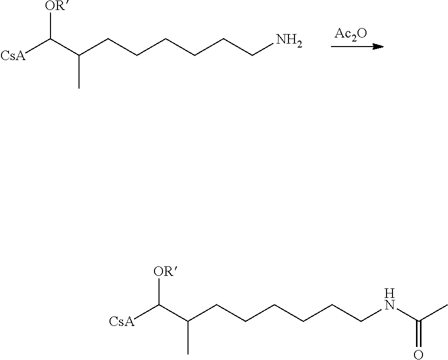

### Reaction 7—Modification of Amino Acid 3

CsA undergoes substitution on AA3 as outlined below.

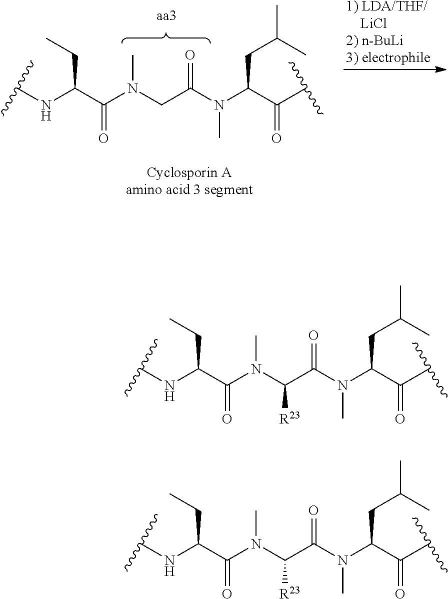

### Route A: [D-MeSar]³-CsA

An oven dried flask is charged under argon atmosphere with 160 mL anhydrous THF and diisopropylamine (2.07 mL, 14.8 mmol). The solution is cooled to −78° C. and n-butyl lithium (2.5 M in hexane, 5.4 mL, 13.5 mmol) is added. After stirring for 30 minutes, CsA (2.40 g, 2.0 mmol, dissolved in 40 mL anhydrous THF) is added. The reaction is stirred for 1 hour at −78° C. Additional n-butyl lithium (3.2 mL, 8.0 mmol) is added, followed by addition of methyl iodide (1.25 mL, 20.0 mmol). Stirring is continued at −78° C. for 1.5 hours, and then the reaction is allowed to warm to room temperature over an additional 1.5 hours. 20 mL H₂O are added and the THF is removed in vacuum. Additional 50 mL H₂O are added and an extraction is carried out with 150 mL EtOAc. The extract is washed with brine and dried over Na₂SO₄. The solvent is removed in vacuum and the crude product is purified over silica gel (hexane/acetone 3:1). Yield: 0.74 g (0.61 mmol, 30%).

### Route B: [MeSar]³-CsA

A dry 100 mL flask is charged under argon atmosphere with 7.5 mL anhydrous THF and diisopropylamine (0.46 mL, 3.3 mmol). The solution is cooled to 0° C. and n-butyl lithium (1.32 mL, 2.5 M solution in hexane, 3.3 mmol) is added. The reaction is stirred for 20 minutes at 0° C. and is then cooled to −78° C. A solution of CsA (601 mg, 0.5 mmol) and lithium chloride (636 mg, 15 mmol) in 12 mL anhydrous THF is prepared and cooled to −78° C. under argon atmosphere. The LDA solution is then transferred into this mixture through a cannula. The reaction is stirred at −78° C. for 2 hours. Additional n-butyl lithium (1.20 mL, 3.0 mmol) is added, followed by methyl iodide (0.62 mL, 10 mmol). The mixture is allowed to warm to −20° C. and stirred at this temperature for 3 hours. The reaction is allowed to warm to room temperature, quenched with saturated NH₄Cl solution, extracted with EtOAc (2×20 mL), washed with brine and dried over Na₂SO₄. The solvent is removed in vacuum and the crude product is purified over silica gel (hexane/acetone 3:1). Yield: [L-MeAla³]-CsA: 302 mg (0.25 mmol, 50%). [D-MeAla³]-CsA: 76 mg (0.06 mmol, 12%).

TABLE 1
Examples of possible electrophiles used for the alkylation of the 3-position of Cyclosporine

| | R²³ (according to | | |
|---|---|---|---|
| Electrophile | Reaction 7) | Route | Remarks |
| methyl iodide | —CH₃ | A/B | — |
| ethyl iodide | —CH₂CH₃ | A/B | — |
| allyl bromide | —CH₂CHCH₂ | B | — |
| 1,3-diiodopropane | —CH₂CH₂CH₂I | B | — |
| 1,4-diiodobutane | —(CH₂)₃CH₂I | B | — |
| trimethylammonium- | —(CH₂)₃N⁺(CH₃)₃ | B | — |
| 3-iodopropane | | | |
| hexafluorophosphate | | | |
| propargyl bromide | —CH₂CCH | A | 10 equiv electrophile; stirred for 1 h |
| | | | at room temperature after |
| | | | electrophile addition |
| tert-butyl | —CH₂CO₂(t-Bu) | A | 5 equiv electrophile; stirred for 1 h |
| bromoacetate | | | at room temperature after |
| | | | electrophile addition |
| benzyl bromide | —CH₂Ph | A | 15 equiv LDA and 30 equiv |
| | | | electrophile; stirred for 6 h at −75° C. |
| | | | and 10 h at room temperature |
| | | | after electrophile addition |
| formaldehyde | —CH₂OH | A | formaldehyde prepared from |
| | | | paraformaldehyde at 170° C. prior |
| | | | to addition |
| acetaldehyde | —CH(OH)CH₃ | B | stirred for 2.5 h at −78° C. after |
| | | | electrophile addition |
| pivalaldehyde | —CH(OH)(t-Bu) | B | stirred for 70 min at −78° C. after |
| | | | electrophile addition |
| benzaldehyde | —CH(OH)Ph | B | stirred for 2.5 h at −78° C. after |
| | | | electrophile addition |
| carbon dioxide | —COOH | A | CO₂ gas passed for 15 min through |
| | | | reaction mixture at −78° C.; stirred |
| | | | for 1 h at −78° C. after electrophile |
| | | | addition |
| dimethyl disulfide | —SCH₃ | B | stirred for 18 h at 0° C. after |
| | | | electrophile addition |
| p-tolyl disulfide | —S(p-Tol) | B | stirred for 18 h at 0° C. after |
| | | | electrophile addition |

Ph = phenyl;
t-Bu = tert-butyl;
Tol = tolyl.

### Reaction 8—AA1 Modification of Alkylated CsA

Following the 3-alkylation, a 2-step procedure leads to the acetylated aldehyde (compound 3 in the example below), which is a suitable substrate for the modification of the 1-position via Wittig reaction. This method allows introduction of residues to the AA1 side-chain that have limited stability under the reaction conditions used in steps 1-3, such as strong base and oxidizing agents.

Further examples of compounds prepared using this sequence is summarized in Table 2.

### Step 1: Alkylation of AA3 Side-Chain

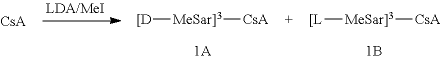

Synthesis is carried out according to Route A or B, respectively, as described above.

### Step 2: Acetylation of the Hydroxy-Group on AA1 Side-Chain

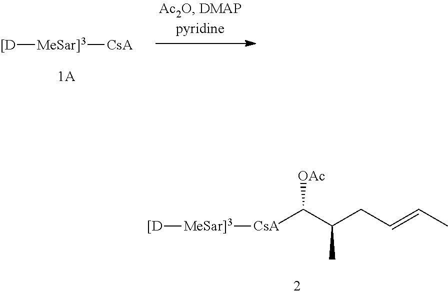

An oven dried flask is charged under nitrogen with [D-MeSar]³-CsA (1.84 g, 1.51 mmol), N,N-dimethylaminopyridine (19 mg, 0.15 mmol) and 20 mL anhydrous pyridine, followed by acetic anhydride (10 mL, 0.1 mol). The reaction is stirred at ambient temperature overnight. The mixture is poured into 100 mL ice-water and is stirred until all ice has melted. A solid is collected by filtration and dried in air. The solid is dissolved in 50 mL EtOAc and is washed with 1 M HCl (2×), sat. NaHCO₃ solution and brine. The organic phase is dried over Na₂SO₄ and evaporated. The crude product is purified over silica gel (hexane/EtOAc/MeOH 10:10:0.5).

### Step 3: Aldehyde Formation

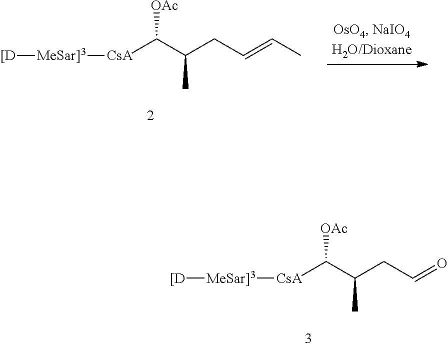

To a flask containing compound 2 (800 mg, 0.636 mmol) are added 10 mL dioxane and 10 mL H₂O. NaIO₄ (544 mg, 2.54 mmol) and OsO₄ (7.9 mM solution in water/dioxane 1:1, 4.05 mL, 32 mmol) are added and the reaction is stirred at room temperature overnight. 75 mL H₂O is added and the reaction is extracted with 3×25 mL EtOAc. The extracts are washed with water, sat. NaHCO₃ solution, water and brine (25 mL each) and are dried over MgSO₄. The solvent is removed in vacuum and the crude product is purified over silica gel (hexane/EtOAc 3:1).

### Step 4: Wittig Reaction

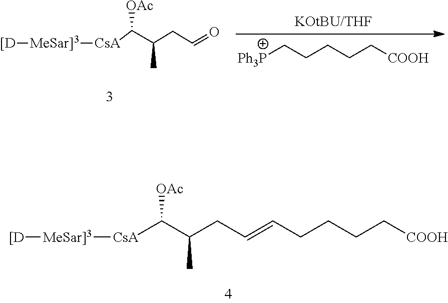

An oven dried flask is charged under argon atmosphere with triphenyl-6-hexanoic acid phosphonium bromide (90 mg, 0.195 mmol) and 5 mL anhydrous THF. Potassium t-butoxide (1 M solution in THF, 0.39 mL, 0.39 mmol) is added at 0° C. and the solution is stirred for 30 minutes to give a bright orange color. Compound 3 (81 mg, 0.065 mmol, dissolved in 1 mL anhydrous THF) is added to the reaction drop-wise and stirring is continued at room temperature overnight. The reaction is quenched with sat. NH₄Cl solution and is extracted with EtOAc. The extract is washed with brine and dried over Na₂SO₄. The solvent is removed in vacuum and the crude product is purified over silica gel (toluene/acetone 3:1).

### Step 5: Deacetylation

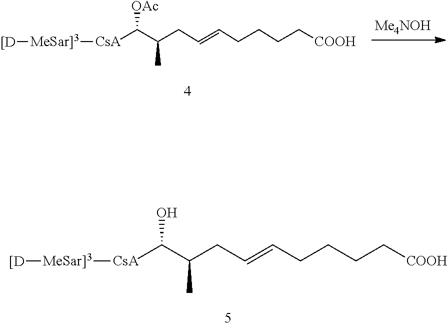

Compound 4 (30 mg, 0.022 mmol) is dissolved in 2 mL methanol and 0.5 mL water and tetramethylammonium hydroxide pentahydrate (12 mg, 0.066 mmol) is added. The reaction is stirred at room temperature for several days until HPLC confirms deprotection is complete. The reaction is acidified to pH 2 with 1 M HCl and concentrated in a vacuum. The residue is taken up in EtOAc, is washed with water and dried over Na₂SO₄. The solvent is evaporated and the crude product is purified by preparative HPLC.

Using the method above, the following compounds were synthesized (X and Y in reference to the above schematic representation; and reference of R in X is to indicate attachment of structure to AA1 of CsA).

TABLE 2

| Compound | X | R²³ | Isomer | MS (Na⁺) |
|---|---|---|---|---|
| 431-13 | 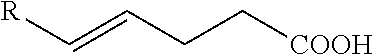 | —CH₃ | L | 1296.8 |
| 414-64 | 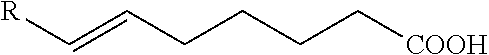 | —CH₃ | D | 1325.0 |
| 431-19 | 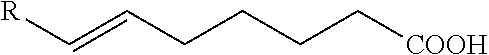 | —CH₃ | L | 1324.8 |
| 431-40 | 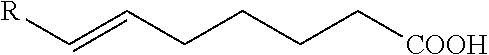 | —CH₂CH₃ | D | 1338.8 |
| 440-02 | 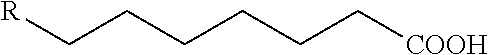 | —CH₃ | D | 1327.1 |
| 431-20 | 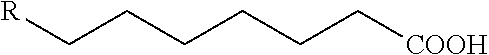 | —CH₃ | L | 1326.9 |
| 440-13 | 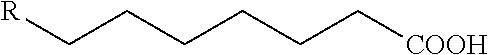 | —CH₂CH₃ | D | 1341.1 |
| 431-21 | 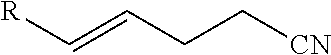 | —CH₃ | D | 1277.9 |
| 431-44 | 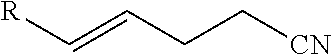 | —CH₂CH₃ | L | 1291.9 |
| 440-14 | 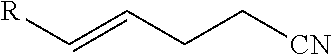 | —CH₂CH₃ | D | 1292.0 |
| 431-136 | 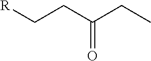 | —CH₃ | L | 1283.1 |
| 440-24 | 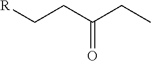 | —CH₃ | D | 1283.1 |
| 440-10-1 | 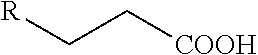 | —CH₃ | D | 1270.9 |
| 440-22-1 | 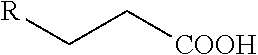 | —CH₂CH₃ | D | 1285.0 |
| 440-20 | 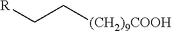 | —CH₃ | D | 1397.2 |

### Reaction 9—Alkylation of AA1 Modified Compounds

Reaction substituents to the AA3 residue of compounds previously modified on the AA1 side-chain. In addition to the groups available through Reaction 7, this route allows the introduction of substituents at AA3 that are unstable under the reaction conditions used in Reaction 8, e.g. a thiomethyl residue could undergo oxidation during the formation of the aldehyde in step 3 of this method.

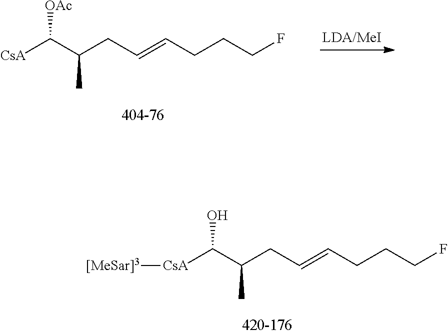

A dry 25 mL flask is charged under argon atmosphere with 1.5 mL anhydrous THF and diisopropylamine (87 μL, 0.62 mmol). The solution is cooled to 0° C. and n-butyl lithium (2.5 M in hexane, 0.25 mL, 0.62 mmol) is added. The mixture is stirred for 20 minutes at 0° C. and is then cooled to −70° C. The clear LDA solution is transferred into a solution of 404-76 (118 mg, 0.095 mmol) and lithium chloride (120 mg, 2.84 mmol) in 1.5 mL anhydrous THF at −70° C. Stirring is continued for 2 hours at −70° C. Additional n-butyl lithium (0.23 mL, 0.58 mmol) is added, followed by methyl iodide (118 μL, 1.89 mmol). The reaction is allowed to warm to −20° C. and is kept at this temperature overnight. The reaction is quenched with sat. NH₄Cl solution and is extracted with EtOAc. The extract is washed with brine, dried over Na₂SO₄ and evaporated to dryness. The crude product is purified over silica gel (hexane/acetone 3:1→2:1).

TABLE 3
Examples of compounds prepared by Reaction 9 (X and R²³ according to the above
schematic representation of 1,3 modified cyclosporine derivatives; and reference of R in X is to
indicate attachment of structure to AA1 of CsA)

| Compound | X | R²³ | Isomer | MS (Na⁺) |
|---|---|---|---|---|
| 420-176-1 | 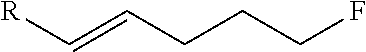 | —CH₃ | D | 1284.9 |
| 420-176-2 | 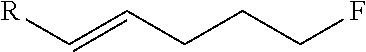 | —CH₃ | L | 1284.9 |
| 420-177-1 | 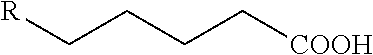 | —CH₃ | D | 1298.8 |
| 420-177-2 | 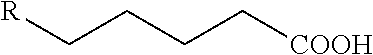 | —CH₃ | L | 1298.8 |
| 420-180-1 | 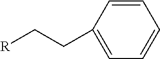 | —CH₃ | D | 1302.8 |
| 420-182-1 | 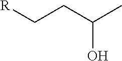 | —CH₃ | D | 1270.8 |
| 420-182-2 | 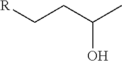 | —CH₃ | L | 1270.8 |
| 420-186 | 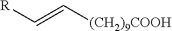 | —CH₂CH₃ | L | 1409.0 |
| 431-42 | 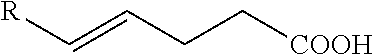 | —SCH₃ | D/L ¹ | 1328.8 |
| 440-03 | 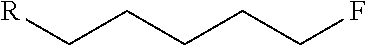 | —SCH₃ | D | 1319.1 |
| 440-78 | 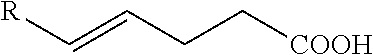 | —(CH₂)₃N⁺(CH₃)₃ | D/L ¹ | 1359.9 ² |
| 440-34-3 | 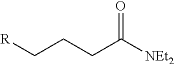 | 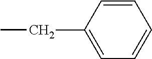 | D | 1416.2 |
| 440-36 | 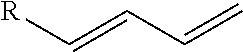 | —CH₃ | D | 1251.1 |

¹ isomers not separated;
² m⁺ signal.

Amide Formation from Nitrite Compound (Reversed Amide)—Synthesis of 440-15

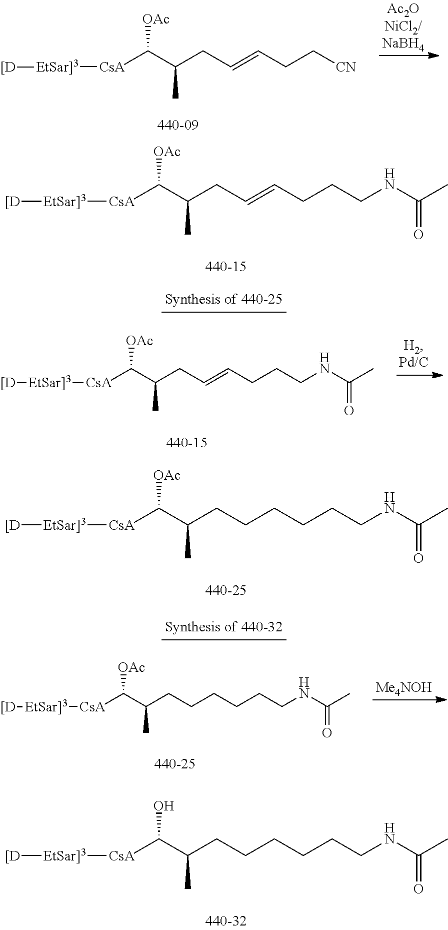

TABLE 4
Examples of 1,3-modified cyclosporine compounds obtained by reducing the double
bond created in the Wittig reaction

| Compound | X | R²³ | Isomer | MS (Na⁺) |
|---|---|---|---|---|
| 431-23 | 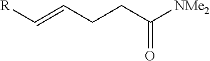 | —CH₃ | L | 1323.8 |
| 431-29 | 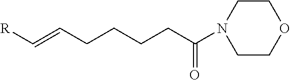 | —CH₃ | L | 1393.9 |
| 440-08 | 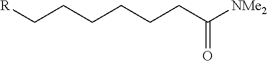 | —CH₃ | D | 1354.1 |
| 440-27 | 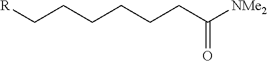 | —CH₂CH₃ | D | 1368.1 |
| 440-23 | 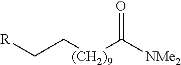 | —CH₃ | D | 1424.2 |
| 431-32 | 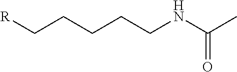 | —CH₃ | D | 1325.9 |
| 431-53 | 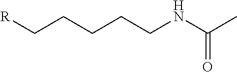 | —CH₂CH₃ | L | 1339.9 |
| 440-32 | 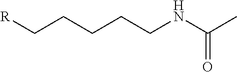 | —CH₂CH₃ | D | 1340.1 |
| 440-10-2 | 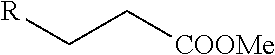 | —CH₃ | D | 1285.0 |
| 440-22-2 | 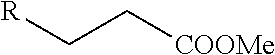 | —CH₂CH₃ | D | 1299.1 |
| 440-31 | 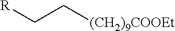 | —CH₃ | D | 1425.2 |

### Example 2

Compound II may be prepared in accordance with known procedures, or variations thereof that will be apparent to those skilled in the art. For example, Compound II may be synthesized using methods analogous to those previously described for 9-S-[3-hydroxy-2-(phosphonomethoxy) propyl]-adenine [(S)-HPMPA] derivatives (see Beadle et al., J. Med. Chem. 49, 2010-2015 (2006); Painter et al., Antimicrob. Agents Chemother. 51, 3505 (2007), and US Patent Application Publication No. 2007/0003516, each of which is incorporated herein by reference in its entirety).

An exemplary method for preparing salts of compounds of the present invention are set forth below. In the following description, all variables are, unless otherwise noted, as defined in the formulas described herein. The following non-limiting descriptions illustrate the general methodologies that may be used to obtain the compounds described herein.

In one embodiment, a salt form of Compound II may be prepared by dissolving Compound II in an appropriate solvent,

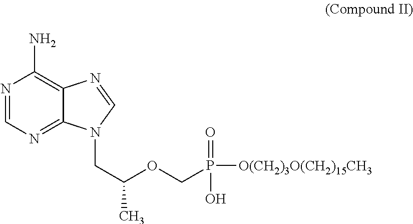

adding a suitable base to the mixture of the solvent and Compound II, and removing the solvent to provide the salts of Compound II.

The solvent used in the preparation may be any suitable solvent known to one skilled in the art or a combination of solvents that provides satisfactory yield of the product. In one embodiment, the solvent is a mixture of at least two solvents. Exemplary combination of solvents includes, but is not limited to, dichloromethane and methanol, dichloromethane and ethanol. In one embodiment, the molar ratio of the dichloromethane and methanol is in a range of about 1:1 to 9:1. In one embodiment, the molar ratio of the dichloromethane and methanol is in a range of about 7:3 to 9:1. In a further embodiment, the molar ratio of the dichloromethane and methanol is about 9:1.

The base used in the preparation may be any suitable base known to one skilled in the art or a combination of bases that provides satisfactory yield of the product. In some embodiments, the base is an alkali metal alcoholate base. Exemplary bases include, but are not limited to, potassium methoxide, sodium methoxide, lithium tert-butoxide, ammonium hydroxide, sodium hydroxide, potassium hydroxide, and lithium hydroxide.

The process described herein may further include the step of recrystallization to remove impurity, side products, and unreacted starting material. The recrystallization step comprises the step of dissolving the product in a suitable solvent at an appropriate temperature, cooling to an appropriate temperature for a sufficient period of time to precipitate the salt of Compound II, filtering to provide the salts of Compound II. In some embodiments, the temperature for the step of dissolving is in a range of about 50° C. to 80° C.

### Example 3. Combination Studies of Compound I with Compound II

The antiviral activity of Compound I and Compound II towards HBV was tested in various cell lines (HepAD38, HepDE19 and HepDES19). The HepAD38 cell line is derived from the HepG2 cell line and engineered to stably maintain HBV (see Ladner et al., Antimicrobial Agents and Chemotherapy, 1997, 41, 1715-1720, which is incorporated herein by reference in its entirety). DE19 and DES19 cell lines are human hepatoma derived cell lines (see Guo et al. J. Virol 2007, 81, 12472-84, which is incorporated herein by reference in its entirety). Active HBV replication in the cells was suppressed by inclusion of tetracycline in the cell culture medium, which binds to a tetracycline-sensitive promoter, and induced by removal of tetracycline. HBV replication was quantified by measuring the amount of HBV DNA in the cells.

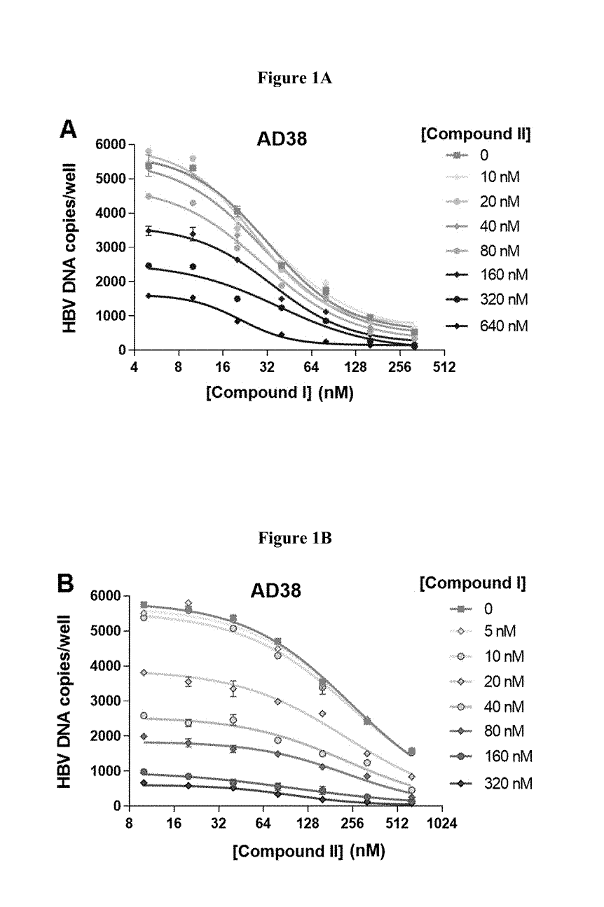

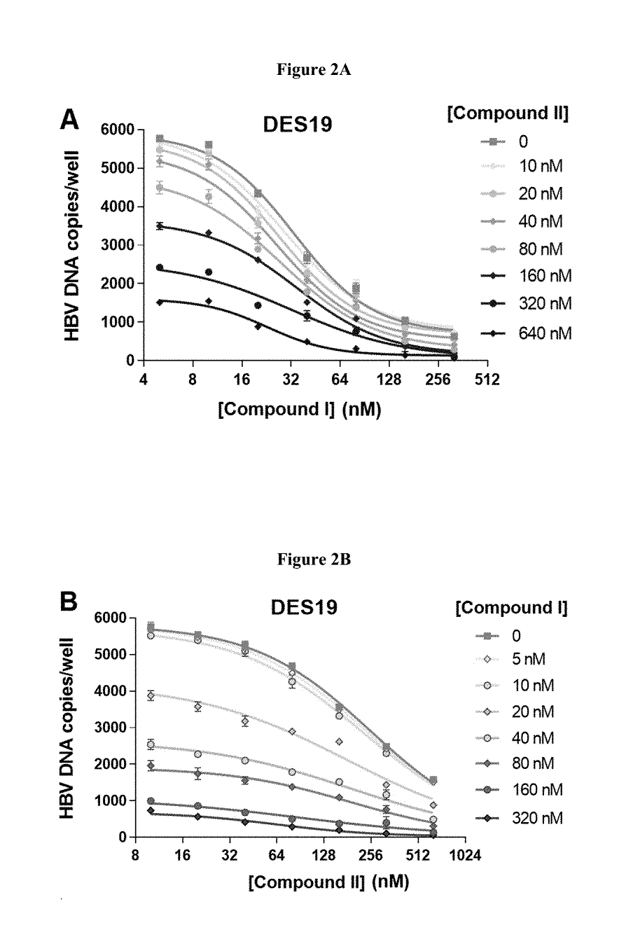

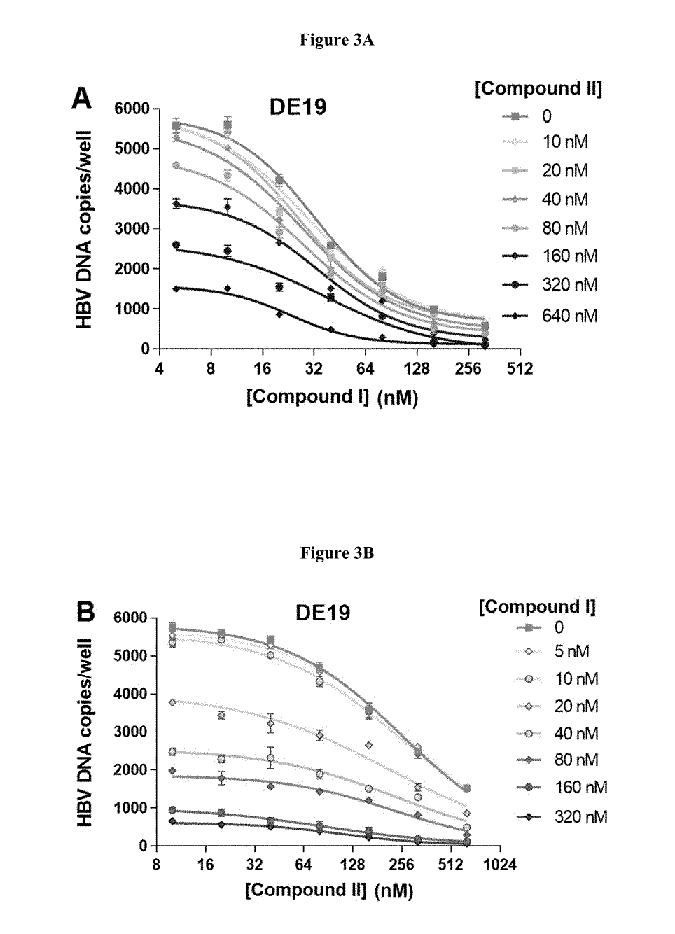

Compound I and Compound II were applied to induced cells (i.e., cells without tetracycline), either alone or in combination at various concentrations. Next, intracellular DNA was isolated and HBV DNA was measured by polymerase chain reaction (PCR). The percent inhibition of HBV DNA was examined over the concentration ranges of 0-320 nM for Compound I and 0-640 nM for Compound II. Concentration versus effect curves were constructed for both drugs (FIGS. 1A and 1B, 2A and 2B, and 3A and 3B). Furthermore, IC₅₀ (inhibitory concentrations that resulted in a 50% inhibition of HBV DNA replication) values were calculated (Tables 6 and 7). As demonstrated in FIGS. 1A, 2A, and 3A (curves where [Compound I]=0), Compound I applied alone in the concentration range of 5-320 nM reduced intracellular HBV DNA in a concentration-dependent manner compared to induced cells in the absence of the drug. Similarly, Compound II applied alone in the concentration range of 10-640 nM reduced intracellular HBV DNA in a concentration-dependent manner compared to induced cells in the absence of the drug. See FIGS. 1B, 2B, and 3B (curves where [Compound II]=0). Combinations of various concentrations of Compound I and Compound II also reduced intracellular HBV DNA (see FIGS. 1 A and 1B, 2A and 2B, and 3A and 3B).

The level of reduction in intracellular HBV DNA by the various combinations of Compound I and II as plotted in FIGS. 1 A and 1B, 2A and 2B, and 3A and 3B, was analyzed using the Shipman-Prichard synergy scoring program (MacSynergy™ II program, Prichard et al. Antiviral Research 1990), wherein the experimentally observed effect of each combination is compared to the theoretical, additive inhibition. The comparison is used to determine combination scores (effect volumes) that represent either synergistic (positive scores) or antagonistic (negative scores) effects. All the positive and negative scores are summed to calculate overall synergy and antagonism scores that reflect the entire concentration spectrum of the two drugs. The magnitude of the scores reflect the degree of synergism/antagonism and the likelihood of the synergistic/antagonistic activities being important in vivo, according to the following four categories: (1) Synergy/Antagonism Volume=0-25 (log volume <2): insignificant synergy/antagonism (i.e., only additive effects); (2) Synergy/Antagonism Volume=25-50 (log volume 2-5): minor but significant amount of synergy/antagonism; (3) Synergy/Antagonism Volume=50-100 (log volume 5-9): moderate synergy/antagonism that may be important in vivo; (4) Synergy/Antagonism Volume >100 (log volume >9): stronger synergy/antagonism that is probably important in vivo. The combination scores obtained as described above are shown in Table 5. IC₅₀ values for Compound I in combination with different concentrations of Compound II, and Compound II in combination with different concentrations of Compound I, are presented in Tables 6 and 7, respectively.

TABLE 5
MacSynergy ™ II drug combination scores for Compound I (5-320 nM)
and Compound II (10-640 nM) in various cell lines

| | Synergy Plot: | | |
|---|---|---|---|
| | (Prichard-Shipman MacSynergyII) | | |
| | 95% confidence interval | | |
| | HepAD38 | DE19 | DES19 |
| SYNERGY | 232.15 | 136.98 | 175.8 |
| 95% confidence interval | 324 to 140 | 222 to 52 | 270 to 82 |
| ANTAGONISM | −22.86 | −13.88 | −3 |
| 95% confidence interval | −6 to −39 | 0 to −27 | Not significant |

TABLE 6
IC₅₀ values for Compound I in AD38, DE19 and DES19 cell
lines, as a function of concentration of Compound II

| | | Compound II (nM) | | | | | | | |
|---|---|---|---|---|---|---|---|---|---|
| | Cell line | 0 | 10 | 20 | 40 | 80 | 160 | 320 | 640 |
| Compound | AD38 | 32.14 | 30.98 | 26.5 | 29.94 | 29.54 | 34.48 | 38.21 | 21.03 |
| I (nM) | DE19 | 32.85 | 29.52 | 25.97 | 27.31 | 27.78 | 33.63 | 37.54 | 23.28 |
| | DES19 | 33.36 | 29.06 | 27.2 | 26.45 | 27.79 | 34.7 | 32.35 | 22.9 |

TABLE 7
IC₅₀ values for Compound II in AD38, DE19 and DES19 cell
lines, as a function of concentration of Compound I

| | | Compound I (nM) | | | | | | | |
|---|---|---|---|---|---|---|---|---|---|
| | Cell line | 0 | 5 | 10 | 20 | 40 | 80 | 160 | 320 |
| Compound | AD38 | 248.1 | 258.6 | 255.3 | 232 | 232.3 | 229.1 | 104.2 | 99.73 |
| II (nM) | DE19 | 253 | 270.3 | 255.2 | 212.6 | 237.2 | 225.7 | 83.94 | 109.6 |
| | DES19 | 252.5 | 236.8 | 227.7 | 180.4 | 189.9 | 188.2 | 86.58 | 63.24 |

Drugs with differing modes of action (MOAs), used in combination, can reasonably be expected to demonstrate additive effects. That is, drug “A” plus drug “B” should reasonably be expected to demonstrate the individual activity or activities of drug A given as monotherapy plus drug B given as monotherapy. However, Compounds I and II tested, in vitro, in combination with each other demonstrated a synergistic effect; an effect greater than would be predicted by either drug given alone.

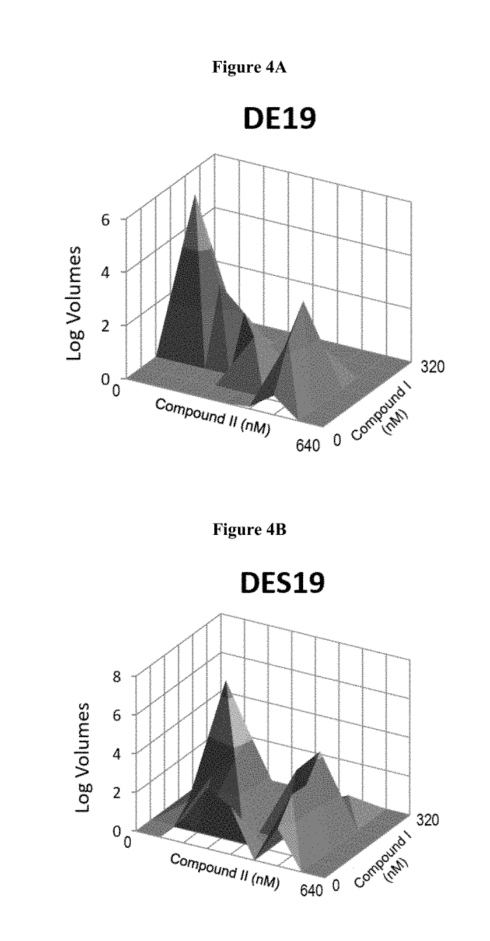

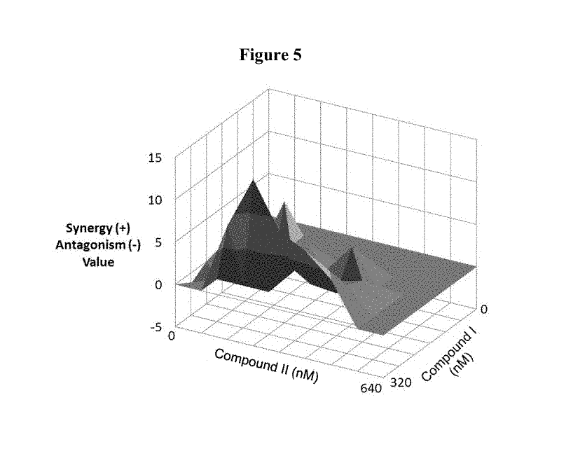

As shown in Table 5, this in vitro combination study of Compound I and Compound II in AD38, DES19, and DE19 cells demonstrated synergy scores ranging from approximately 110 to 230 in a first study and, in a second separate study, scores ranging from about 20 to 80. These scores all indicated synergy, ranging from mild synergy to strong synergy. Furthermore, no significant antagonistic effects are found when combining Compound I and Compound II. The synergy plots for all three cell lines are represented in FIGS. 4A, 4B, and 5.

The concrete results of synergism provides possible advantages of combination of Compound I and Compound II as anti-HBV and anti-HIV therapies. For example, it is possible to significantly reduce HBV or HIV viral load with a relatively low drug burden via the combination of Compound I and Compound II (e.g., reduced number of dosages and/or reduced strength of dosage), which in turn can reduce off-target effects (e.g., nephrotoxicity, hepatotoxicity, cardiac dysfunction). The mechanisms responsible for the synergistic effects on HBV or HIV DNA may also contribute to inhibiting other markers of HBV or HIV viral load or stages of the HBV or HIV life cycle, thereby leading to a more effective anti-HBV or anti-HIV therapy.

### Example 4—Independent and Combinational Anti-HBV Effects of Compound I and Compound II in the HBV Transgenic Mouse Model

### Purpose

The purpose of this study was to investigate the effect(s) of Compound I and Compound II, administered alone and in combination, in the HBV transgenic mouse model.

### Methods

Compound I and Compound II were administered by once-daily oral gavage for 16 days to HBV transgenic mice which replicated HBV from a 1.3× overlength HBV genome integrated into the mouse genome. Expression occurred mostly in hepatocytes.

Treatment Groups (n=8/Group):

a) Vehicle

b) Compound I low dose−10 mg/kg/day

c) Compound I high dose−50 mg/kg/day

d) Compound II low dose−5 mg/kg/day

e) Compound II high dose−10 mg/kg/day

f) Compound I low dose (10)+Compound II low dose (5)

After 16 days of dosing, livers and serum were harvested and the following parameters were measured:

a) HBV DNA by quantitative PCR (liver and serum)

b) HBsAg by ELISA (liver and serum)

c) HBeAg by ELISA (serum)

Compound I Concentrations were Measured by Liquid Chromatography-Mass Spectrometry (Serum)

### Results

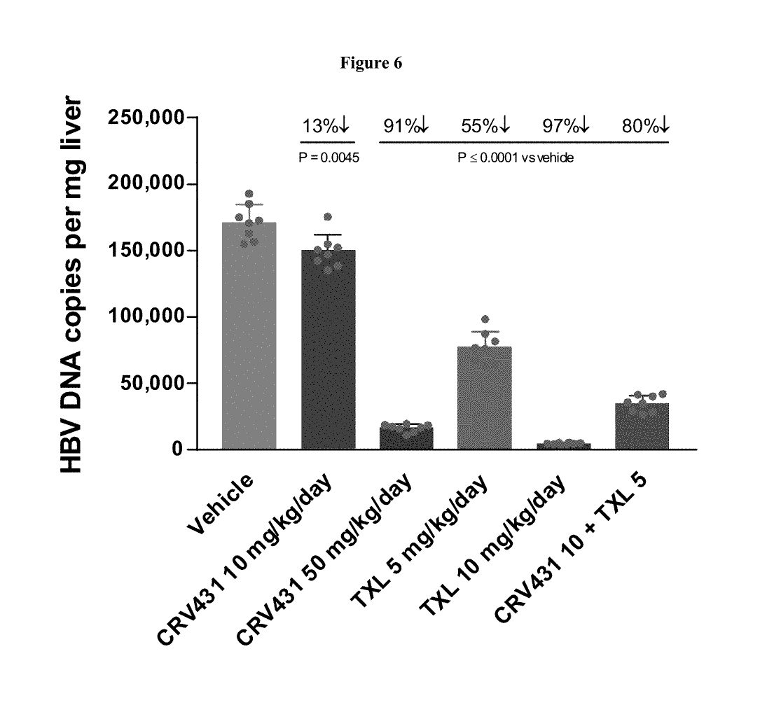

FIG. 6 shows HBV DNA copies per mg liver after treatment with vehicle, Compound I and/or Compound II. As shown in FIG. 6, Compound I administered alone decreased liver HBV DNA. Compound II administered alone decreased liver HBV DNA. The combination of Compound I and Compound II decreased HBV DNA more than either drug alone.

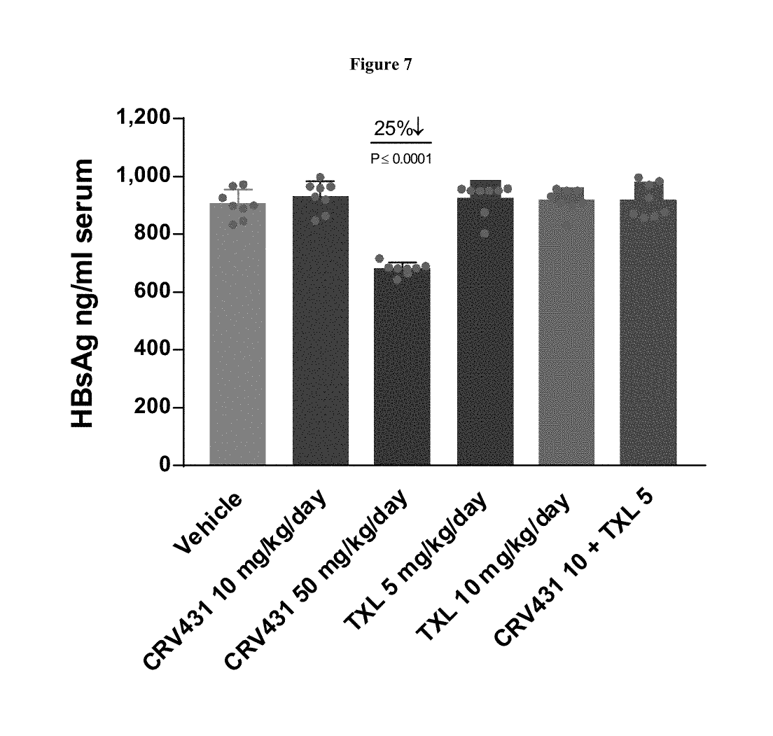

FIG. 7 shows serum HBsAg levels after treatment with vehicle, Compound I and/or Compound II. As shown in FIG. 7, high-dose Compound I decreased serum HBsAg. Compound II administered alone or in combination with low-dose Compound I did not affect serum HBsAg.

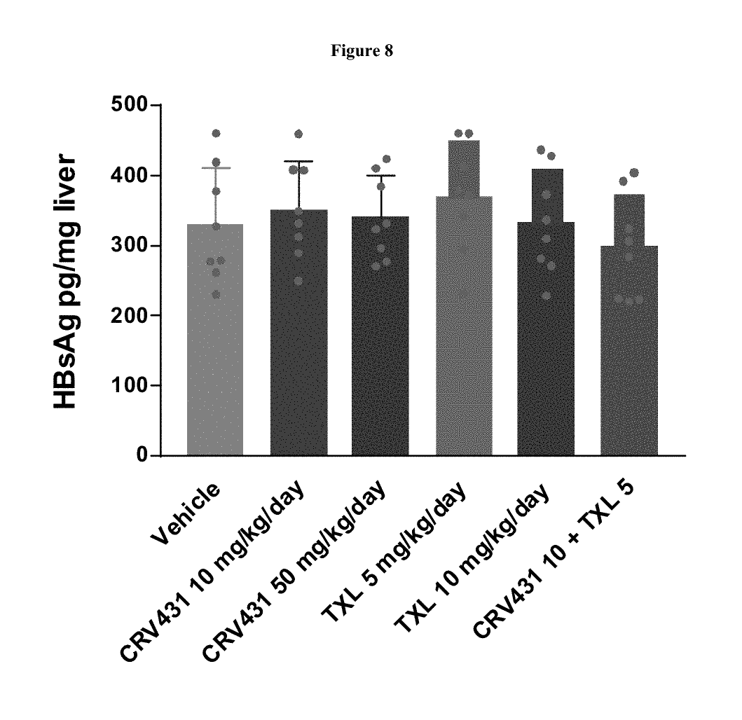

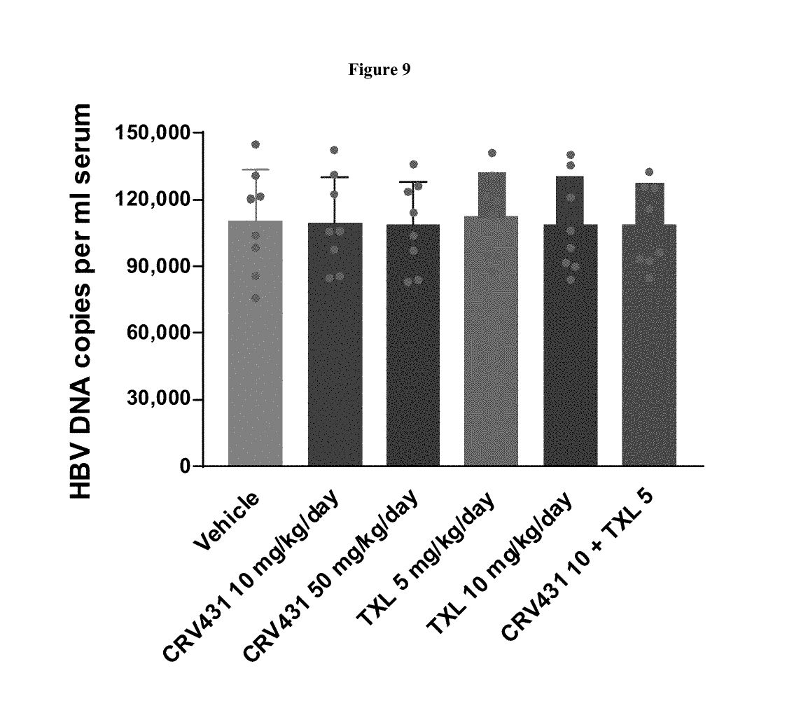

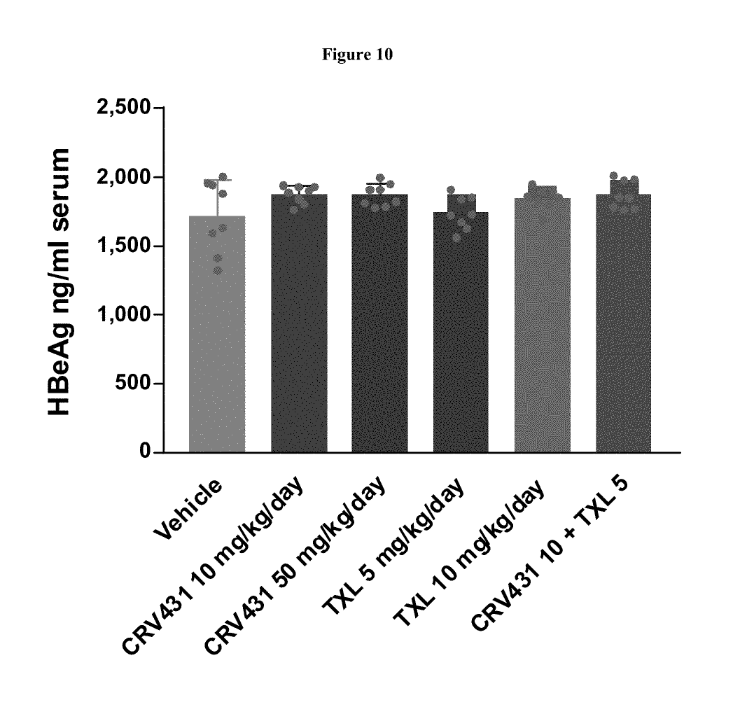

FIGS. 8, 9 and 10 show plots for liver HBsAg, serum HBV DNA, and serum HBeAg levels, respectively. As shown in FIGS. 8, 9 and 10, Compound I and Compound II, alone or in combination, did not affect levels of liver HBsAg, serum HBV DNA, or serum HBeAg.

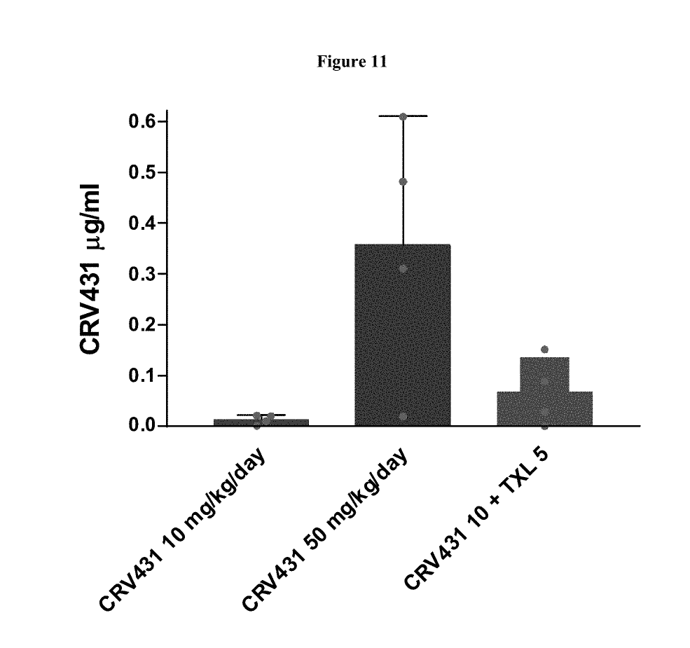

FIG. 11 shows the measured levels of Compound I in serum. As shown in FIG. 11, the serum Compound I concentration at 3-hr post-dose on Day 16 of dosing correlated with the effects of Compound I on HBV DNA and HBsAg.

Compound I and Compound II were both highly efficacious as monotherapies at lowering HBV DNA in the livers of transgenic mice. Unlike nucleoside or nucleotide analog therapies (including Compound II in the present study), Compound I was able to lower serum HBsAg levels.

Additive or greater reductions in HBV DNA were achieved with Compound I and Compound II combination treatment.

### EQUIVALENTS

Those skilled in the art will recognize, or be able to ascertain using no more than routine experimentation, many equivalents to the specific embodiments and methods described herein. Such equivalents are intended to be encompassed by the scope of the present application. All patents, patent applications, and literature references cited herein are hereby expressly incorporated by reference.

1. A method of treating or preventing a hepatitis B virus (HBV) disease or a human immune deficiency (HIV) disease, comprising administering to a subject in need thereof a compound of Formula I: 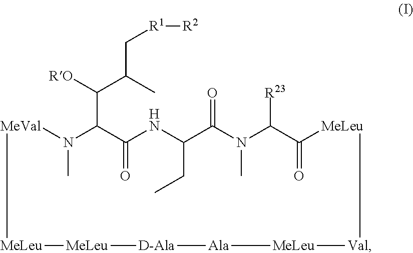
or a pharmaceutically acceptable salt thereof, wherein:
R′ is H or acetyl;
R¹ is a saturated or unsaturated straight or branched aliphatic carbon chain from 2 to 15 carbon atoms in length;
R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;
wherein the saturated or unsaturated straight or branched aliphatic carbon chain is optionally substituted with the aromatic group;
wherein the aromatic group is optionally substituted with the saturated or unsaturated straight or branched aliphatic carbon chain; and
R²³ is a saturated or unsaturated straight or branched optionally substituted aliphatic carbon chain;
in combination with a nucleoside analog reverse transcriptase inhibitor or a nucleotide analog reverse transcriptase inhibitor.

2. The method of claim 1, wherein:
R′ is H;
R¹-R² is 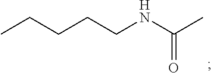
and
R²³ is methyl.

3. The method of claim 1, wherein the chiral center to which R²³ is attached is in the (R) configuration.

4. The method of claim 3, wherein the compound of Formula I is:

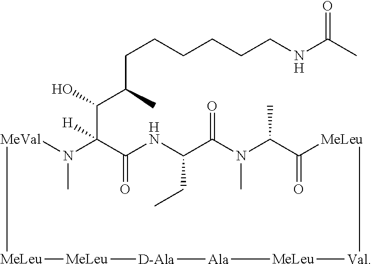

5. The method of claim 1, wherein the compound of Formula I, or a pharmaceutically acceptable salt thereof, and the nucleoside analog reverse transcriptase inhibitor or the nucleotide analog reverse transcriptase inhibitor, are administered simultaneously.

6. The method of claim 1, wherein the compound of Formula I, or a pharmaceutically acceptable salt thereof, and the nucleoside analog reverse transcriptase inhibitor or the nucleotide analog reverse transcriptase inhibitor, are administered sequentially.

7. The method of claim 6, wherein the compound of Formula I, or a pharmaceutically acceptable salt thereof, is administered prior to the nucleoside analog reverse transcriptase inhibitor or the nucleotide analog reverse transcriptase inhibitor.

8. The method of claim 6, wherein the nucleoside analog reverse transcriptase inhibitor or the nucleotide analog reverse transcriptase inhibitor is administered prior to the compound of Formula I, or a pharmaceutically acceptable salt thereof.

9. The method of claim 1, wherein the compound of Formula I, or a pharmaceutically acceptable salt thereof, and the nucleoside analog reverse transcriptase inhibitor or the nucleotide analog reverse transcriptase inhibitor, are administered in alternation.

10. The method of claim 1, wherein the compound of Formula I, or a pharmaceutically acceptable salt thereof, and the nucleoside analog reverse transcriptase inhibitor or the nucleotide analog reverse transcriptase inhibitor, are comprised in the same dosage unit form.

11. The method of claim 1, wherein at least one of the compound of Formula I, or a pharmaceutically acceptable salt thereof;
and the nucleoside analog reverse transcriptase inhibitor or the nucleotide analog reverse transcriptase inhibitor;
are administered orally.

12-13. (canceled)

14. The method of claim 1, wherein the compound of Formula I, or a pharmaceutically acceptable salt thereof, is administered in an amount from about 1 mg per day to about 1,000 mg per day.

15. (canceled)

16. The method of claim 1, wherein the nucleoside analog reverse transcriptase inhibitor or the nucleotide analog reverse transcriptase inhibitor is administered in an amount from about 5 mg per day to about 400 mg per day.

17. The method of claim 1, wherein at least one of the compound of Formula I, or a pharmaceutically acceptable salt thereof;
and the nucleoside analog reverse transcriptase inhibitor or the nucleotide analog reverse transcriptase inhibitor;
are administered once, twice, or at least three times daily; once every two days, once every three days, or once weekly.

18-19. (canceled)

20. The method of claim 1, wherein at least one of the compound of Formula I, or a pharmaceutically acceptable salt thereof;
and the nucleoside analog reverse transcriptase inhibitor or the nucleotide analog reverse transcriptase inhibitor;
are administered for 1, 2, 3, 4, 5, 6, or 7 days a week.

21-24. (canceled)

25. The method of claim 1, wherein the compound of Formula I, or a pharmaceutically acceptable salt thereof, and the nucleoside analog reverse transcriptase inhibitor or the nucleotide analog reverse transcriptase inhibitor, are administered with the same dosing frequency.

26-27. (canceled)

28. The method of claim 1, wherein the compound of Formula I, or a pharmaceutically acceptable salt thereof, and the nucleoside analog reverse transcriptase inhibitor or the nucleotide analog reverse transcriptase inhibitor, are administered with the same treatment duration.

29. (canceled)

30. The method of claim 1, wherein the nucleoside analog reverse transcriptase inhibitor or the nucleotide analog reverse transcriptase inhibitor and the compound of Formula I are administered in temporal proximity.

31-37. (canceled)

38. A method of modulating HBV or HIV replication, comprising administering to a subject in need thereof a compound of Formula I: 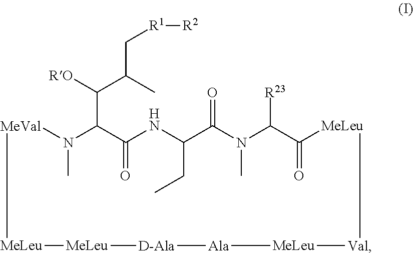
or a pharmaceutically acceptable salt thereof, wherein:
R′ is H or acetyl;
R¹ is a saturated or unsaturated straight or branched aliphatic carbon chain from 2 to 15 carbon atoms in length;
R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;
wherein the saturated or unsaturated straight or branched aliphatic carbon chain is optionally substituted with the aromatic group;
wherein the aromatic group is optionally substituted with the saturated or unsaturated straight or branched aliphatic carbon chain; and
R²³ is a saturated or unsaturated straight or branched optionally substituted aliphatic carbon chain;
in combination with a nucleoside analog reverse transcriptase inhibitor or a nucleotide analog reverse transcriptase inhibitor.

39-45. (canceled)

46. The method of claim 1, wherein the nucleoside analog reverse transcriptase inhibitor or nucleotide analog reverse transcriptase inhibitor is: 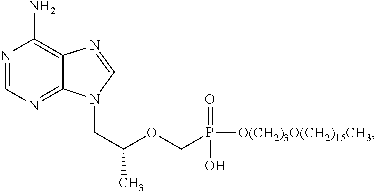
or a pharmaceutically acceptable salt thereof.

47. The method of claim 46, wherein the pharmaceutically acceptable salt of the compound the nucleoside analog reverse transcriptase inhibitor or nucleotide analog reverse transcriptase inhibitor is: 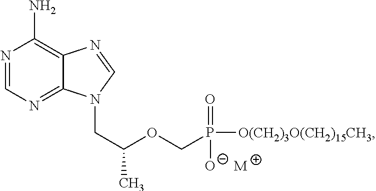
wherein M⁺ is K⁺.

48-60. (canceled)

61. A kit for the treatment or prevention of a HBV disease or a HIV disease, or modulation of HBV of HIV replication, comprising a first container comprising a compound of Formula I: 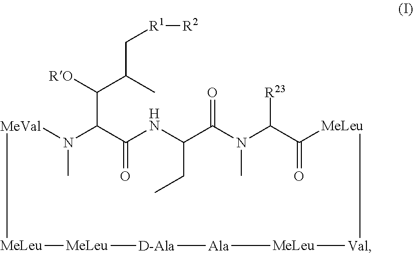
or a pharmaceutically acceptable salt thereof, wherein:
R′ is H or acetyl;
R¹ is a saturated or unsaturated straight or branched aliphatic carbon chain from 2 to 15 carbon atoms in length;
R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;
wherein the saturated or unsaturated straight or branched aliphatic carbon chain is optionally substituted with the aromatic group;
wherein the aromatic group is optionally substituted with the saturated or unsaturated straight or branched aliphatic carbon chain; and
R²³ is a saturated or unsaturated straight or branched optionally substituted aliphatic carbon chain;
and a second container comprising a nucleoside analog reverse transcriptase inhibitor or a nucleotide analog reverse transcriptase inhibitor.

62. A pharmaceutical composition comprising a compound of Formula I: 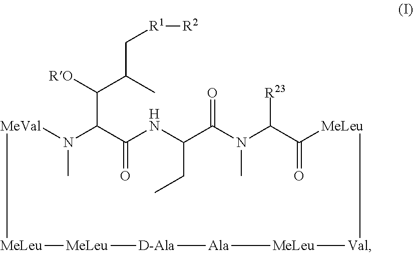
or a pharmaceutically acceptable salt thereof, wherein:
R′ is H or acetyl;
R¹ is a saturated or unsaturated straight or branched aliphatic carbon chain from 2 to 15 carbon atoms in length;
R² is selected from the group consisting of: H; an unsubstituted, N-substituted, or N,N-disubstituted amide; a N-substituted or unsubstituted acyl protected amine; a carboxylic acid; a N-substituted or unsubstituted amine; a nitrile; an ester; a ketone; a hydroxy, dihydroxy, trihydroxy, or polyhydroxy alkyl; a substituted or unsubstituted aryl; a saturated or unsaturated straight or branched aliphatic carbon chain optionally containing a substituent selected from the group consisting of ketones, hydroxyls, nitriles, carboxylic acids, esters, 1,3-dioxolanes, halogens, and oxo; an aromatic group containing a substituent selected from the group consisting of halides, esters, and nitro;
wherein the saturated or unsaturated straight or branched aliphatic carbon chain is optionally substituted with the aromatic group;
wherein the aromatic group is optionally substituted with the saturated or unsaturated straight or branched aliphatic carbon chain; and
R²³ is a saturated or unsaturated straight or branched optionally substituted aliphatic carbon chain;
and a nucleoside analog reverse transcriptase inhibitor or a nucleotide analog reverse transcriptase inhibitor.

63-68. (canceled)
